(19) United States
(12) Patent Application Publication
(10) Pub. No.: US 2020/0001294 A1
(43) Pub. Date: Jan. 2, 2020

(54) LIQUID DISTRIBUTION DEVICE

(71) Applicant: KimanTech, L.L.C., Salt Lake City, UT (US)

(72) Inventors: Nils Adey, Salt Lake City, UT (US); Robert Parry, Salt Lake City, UT (US); Dale Emery, Salt Lake City, UT (US); Derek Bosh, Herriman, UT (US)

(21) Appl. No.: 16/564,797

(22) Filed: Sep. 9, 2019

Related U.S. Application Data
(63) Continuation-in-part of application No. 16/491,539.
(60) Provisional application No. 62/545,317, filed on Aug. 14, 2017.
(60) Provisional application No. 62/564,773, filed on Sep. 28, 2017.
(60) Provisional application No. 62/579,050, filed on Oct. 30, 2017.

(51) Int. Cl. B01L 3/00

(52) U.S. Cl. CPC B01L 3/50273; B01L 3/502746; A61B 2050/0083; B01L 2300/0867; B01L 2300/087; B01L 2200/0621

(57) ABSTRACT

A device for forming a liquid aliquot, the device including: a first layer; an elastic second layer overlapping the first layer; a first passageway to receive and hold a volume of liquid, the first passageway formed from the first and second layers; a first actuator to press on the elastic layer thereby dividing the liquid filled passageway into a series of liquid aliquots; a series of vents associated with the series of aliquots; a second actuator to control flow of liquid aliquots through the associated vents; and an attachment structure for attachment of aliquot receptacles to receive liquid aliquots that flow through the vents.

### BACKGROUND

Polymerase Chain Reaction (“PCR”) revolutionized the processing of DNA. PCR is a powerful and widely used method to identify and obtain a specific DNA sequences from a highly complex sample mixture of DNA. Since the development of PCR, there have been ongoing efforts to improve the information obtained by PCR. These include quantitative PCR (“qPCR”), also known as real-time PCR, which allows determination of concentrations of the nucleic acids in a sample, multiplex PCR which allow multiple targets to be amplified simultaneously in the same vessel, and nested PCR, which allow increased specificity due to increased amplification stringency.

Quantitative PCR (qPCR) involves estimating the initial concentration of a DNA sequence by following the increase in a fluorescence signal as a function of the PCR cycle number. Some types of qPCR, for example non-hydrolysis qPCR, may be followed by melting analysis of the PCR products to help confirm identity. End point PCR involves analyzing the products of the PCR reaction, such as the size of the products by gel electrophoresis, and/or by melting analysis.

Multiplex PCR involves using multiple pairs of PCR primers (an amplicon) in the same reaction vessel such that multiple different amplification products (and thus more information) can be generated from a single aliquot of the sample. This is particularly useful when the sample input is limited. A disadvantage of multiplex PCR is reduced specificity due the increased possibility of unintended PCR products, and reduced sensitivity of the less efficient PCR amplicons.

Nested PCR involves two sequential rounds of PCR where the amplification products of the first (primary) PCR reaction are used as the template for the second (secondary) PCR reaction utilizing a second set of primers (these primer binding sites are located internally on the PCR target of the first set of primers). The product of the primary PCR may be diluted and distributed to multiple secondary tubes before running the secondary PCR. One advantage of nested PCR is increased specificity. A disadvantage of nested PCR is PCR product contamination due to the need to open the primary PCR reaction tube and perform the dilutions.

### SUMMARY OF THE INVENTION

The system described herein is intended to be used to dilute and distribute samples, such as PCR product. In particular, the system facilitates laboratory manipulations and reduces contamination when performing nested PCR and/or other biochemical and chemical processes where a target sequence is initially amplified, diluted, and then amplified a second time. The system interfaces with standardized PCR reagents, consumables, and equipment found in most PCR facilities.

### BRIEF DESCRIPTION OF THE DRAWINGS

The accompanying drawings illustrate various examples of the principles described herein and are a part of the specification. The illustrated examples do not limit the scope of the claims.

FIG. 1a shows a top (plan) view of a device for mixing and distributing liquid according to one example consistent with this specification.

FIG. 1b shows a side (profile) view of the device of FIG. 1A.

FIGS. 1c-1p show alternate embodiments of devices for mixing and distributed liquid according to examples consistent with this specification

FIG. 2a shows the interaction of a port with a tube, which could be a transfer vessel, and a sleeve according to an example consistent with this specification.

FIG. 3 shows a side (profile) view of a device with output tubes attached to the device according to an example consistent with this specification.

FIG. 4 shows a side (profile) view of a three layer device consistent with this specification.

FIG. 5 shows a top (plan) view of a device according to an example consistent with this specification.

FIGS. 6a-6j show a series of operations demonstrating a method of using an example device, shown in FIG. 1, consistent with this specification.

FIG. 7a shows a top (plan) view of a device for mixing and distributing liquid into an array of wells found in a common plate format according to one example consistent with this specification.

FIG. 7b shows a side (profile) view of the device of FIG. 7A.

FIGS. 8a-8d show a series of operations demonstrating a method of using an example device, shown in FIG. 7, consistent with this specification.

FIGS. 9a-9b show a device for implementing the method outlined in FIGS. 8a-d.

FIGS. 10a-10h show a series of operations demonstrating a method of using an example device, shown in FIGS. 1c through 1h, consistent with this specification.

FIGS. 11a-11k show a series of operations demonstrating a method of using an example device shown in FIGS. 1i-1p.

FIG. 12a-12h show a series of operations demonstrating a method of using an example device, shown in FIGS. 1c through 1h.

FIG. 13 shows a device for forming a liquid aliquot according an example consistent with this specification.

FIGS. 14a-14b show a device for forming liquid aliquots consistent with this specification.

FIG. 15 shows a system for forming liquid aliquots consistent with this specification.

FIG. 16 shows a flowchart for a method of forming aliquots of liquid containing nucleic material consistent with this specification.

FIG. 17a shows a side view of the separate components used to build one example of the device.

FIG. 17b shows a side view of the assembled components used to build one example of the device.

FIG. 17c shows a top view of the components used to build one example of the device.

FIG. 18a shows a side view of the assembled components of one example of the device prior to attachment to a well plate.

FIG. 18b shows a side view of the assembled components used to build one example of the device.

FIG. 18c shows a side view of the assembled components of one example of the device after attachment to a well plate.

FIG. 19a shows a top view of one example of the device with one input bladder that can interface with a well plate.

FIG. 19b shows a side view of one example of the device with one input bladder that can interface with a well plate.

FIG. 19c shows a top view of one example of the device with two input bladders that can interface with a single well plate.

FIG. 20a thru 27c show the steps involved with operating the device shown in FIGS. 19a and 19b.

Throughout the drawings, identical reference numbers designate similar, but not necessarily identical, elements. The figures are not necessarily to scale, and the size of some parts may be exaggerated to more clearly illustrate the example shown. Moreover the drawings provide examples and/or implementations consistent with the description; however, the description is not limited to the examples and/or implementations provided in the drawings.

### DETAILED DESCRIPTION

Several difficulties are reduced by the system described herein. These include: PCR product contamination and the rate of false positives. The system also reduces and/or eliminates the need for manually distributing the samples to the output vials, reducing the operator variability in measurement from using a micropipette and/or similar device.

Regarding PCR product contamination, effective operations depend on preventing the products of a primary PCR reaction from contaminating subsequent PCR reactions. If contamination occurs, the signal generated maybe from the contaminating products and not from the sample of interest. Keeping PCR products contained within the reaction vessel is one method to prevent PCR product contamination. Another method to minimize PCR product contamination is to utilize deoxyuridine triphosphate (dUTP) in place of deoxythymidine triphosphate (dTTP) in the PCR reaction. The resulting uracil (U) containing PCR products can be selected against in subsequent PCR reactions by treatment with uracil DNA glycosylase, which degrades U containing DNA templates, but not the normal T containing templates. Performing nested PCR carries a risk of PCR product contamination as the PCR products may not be continuously contained and uracil selection can be used in one of the two rounds of PCR.

Multiplex reactions greatly increase the chances of generating false positive signals due to unintended priming. In multiplex reactions, some amplicons are often favored over others. The number of resolvable fluorescent signals limits the number of amplicons that can be used in multiplex qPCR.

One method of reducing these issues is performing a multiplex primary PCR reaction (often a limited number of cycles, such as 12 to 15), followed by dilution and aliquoting the PCR products, then performing separate nested secondary endpoint PCR and/or qPCR reactions (in separate vessels). This approach allows effective use of a limited sample and provides high specificity.

In an example, the device described here is a plastic consumable that interfaces with a supporting instrument. The device is intended to perform the following operations and give the following advantages: dilute and transfer a primary PCR product from a standard PCR vessel (tubes/tube strips) into multiple secondary PCR tubes and/or tube strips and/or plates of tubes, while keeping the PCR products continuously contained and thereby greatly reducing the chance of PCR product contamination.

One use of this device is to dilute the products of a primary multiplex PCR reaction, then aliquot portions of this liquid into multiple secondary PCR reaction vessels intended for nested PCR. The disclosed examples reduce the possibility of contamination compared with previous techniques. The disclosed examples may allow increased automation. This may reduce the touch time for personnel running the testing, increasing their efficiency. This may also reduce operator to operator variation in performing the test methods, and/or reduce failures and/or mix ups due to mislabeling and/or pipetting errors.

By interfacing with standard format PCR vessels, this device allows the utilization of standard robotics, PCR/qPCR instruments, and reagents already found in most existing PCR facilities. This reduces the cost of use and the cost of adoption. Other devices have been described and developed that support multiplex nested PCR while containing the PCR products. These devices utilize custom non-interchangeable formats that use dedicated PCR/qPCR instruments and reagents. This increases the expense and limits flexibility for many PCR facilities.

In this specification and the associated claims, the term “aliquot” should be understood as a volume of a solution. Multiple aliquots may have equal volumes. Different aliquots may have different volumes. The system may form aliquots of different volumes simultaneously. The system may form a set of uniform volume aliquots.

Among other examples, this specification describes a device for forming a liquid aliquot, the device comprising a first layer and an elastic second layer overlapping the first layer; a first passageway to receive and hold a volume of liquid, the first passageway formed from attachment of the first and second layers; a first actuator to press on the elastic layer thereby dividing the liquid filled passageway into a series of liquid aliquots; a series of vents associated with the series of aliquots; a second actuator to control flow of liquid aliquots through the associated vents; and an attachment structure for attachment of aliquot receptacles to receive liquid aliquots that flow through the vents. This specification also describes a method of separating a liquid containing nucleic material into aliquots, the method including: flowing the liquid through series of linked volumes between two sheets of polymer; and isolating the linked volumes from each other using at least one of: heat and pressure.

This specification describes among other examples, systems for forming aliquots of liquid which minimize the potential for contamination of the aliquots. The systems may interface with an input tube and/or vessel. The systems may provide the aliquots to receiving tubes, a tube strip, a tube plate, and/or a well plate. The systems may flush liquid through the system to clear any air, dilute the sample, mix the sample, etc. The systems may block side passageways leading to the receiving vessels, tubes, and/or wells while flushing the passageway. The passageway is filled with the sample to be divided into aliquots. The passageway is then blocked to form isolated volumes of liquid. The liquid in the isolated volumes is transferred into the receiving vessels, tubes, wells, etc. The liquid may be expressed into the receiving vessels, tubes, wells, etc. Expressing the liquid may involve unblocking the side passageways to allow the liquid to flow to the desired containers. The receiving vessels and a portion of the device sealing them are separated from the rest of the device to allow subsequent processing of the aliquots of liquid.

This specification also describes a device for forming a liquid aliquot, the device including: a first layer; an elastic second layer overlapping the first layer; a first passageway to receive and hold a volume of liquid, the first passageway formed from the first and second layers; a first actuator to press on the elastic layer thereby dividing the liquid filled passageway into a series of liquid aliquots; a series of vents associated with the series of aliquots; a second actuator to control flow of liquid aliquots through the associated vents; and an attachment structure for attachment of aliquot receptacles to receive liquid aliquots that flow through the vents.

Also described is a system for preparing a liquid aliquot, the system including: an elastic passageway, the passageway connected at one end to an input reservoir; a third actuator to control the flow of a liquid from the input reservoir into the passageway; a first actuator to divide the passageway into a plurality of liquid aliquots; and a second actuator to control the flow of a liquid aliquot from an isolated portion of the passageway through a vent and into an attached aliquot receptacle.

Among other examples, this specification describes a method of separating a liquid containing nucleic material into aliquots, the method including: flowing the liquid into an elastic passageway; dividing the passageway using at least one of: heat and pressure, thereby creating a series of liquid aliquots; and dispensing the liquid aliquots through associated vents into individual aliquot receptacles.

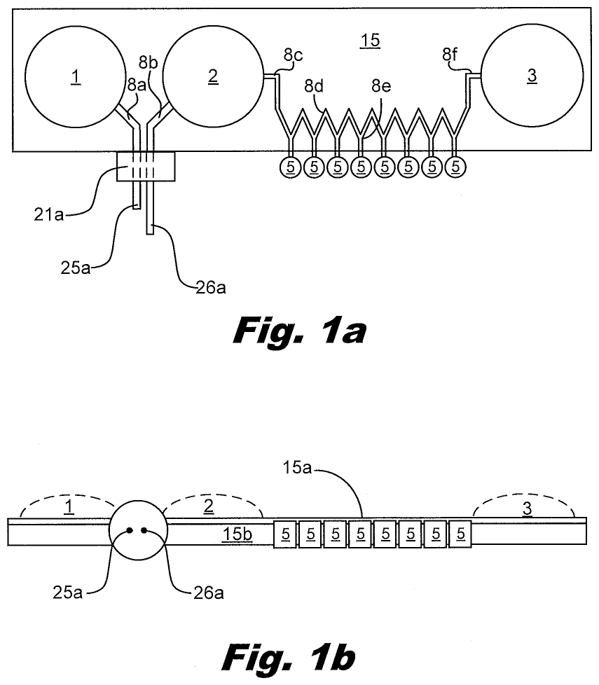

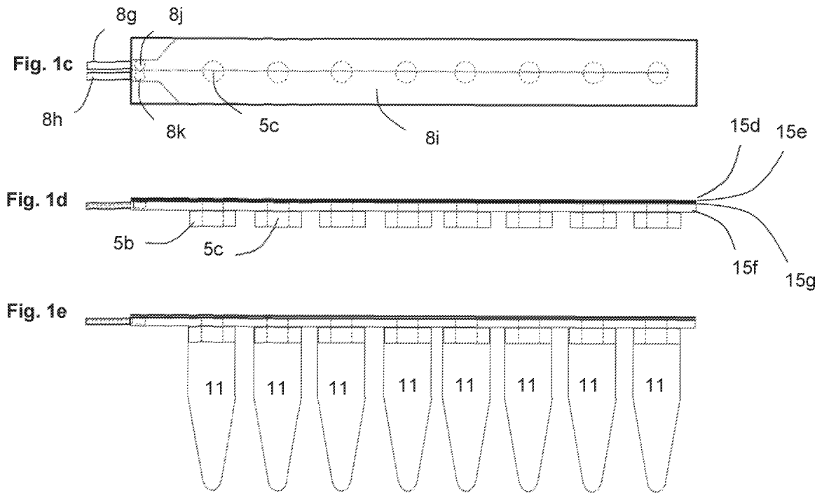

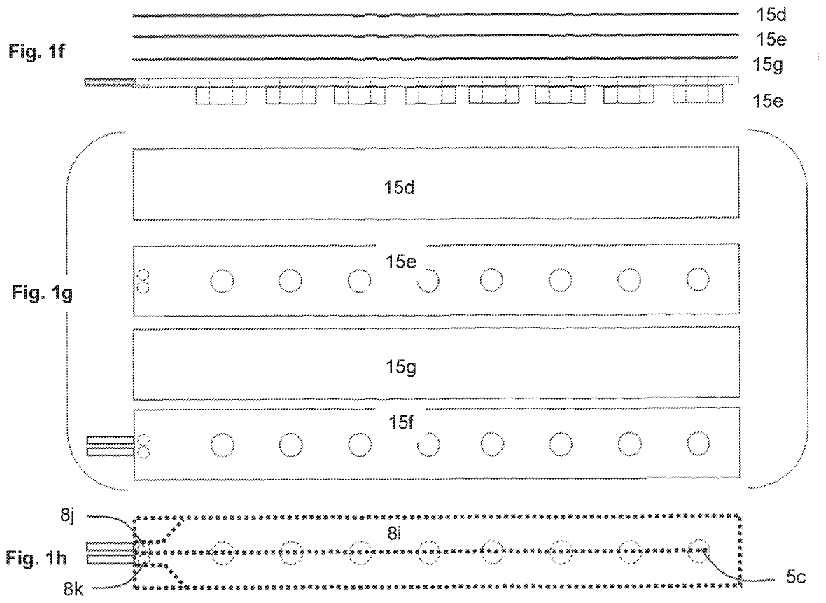

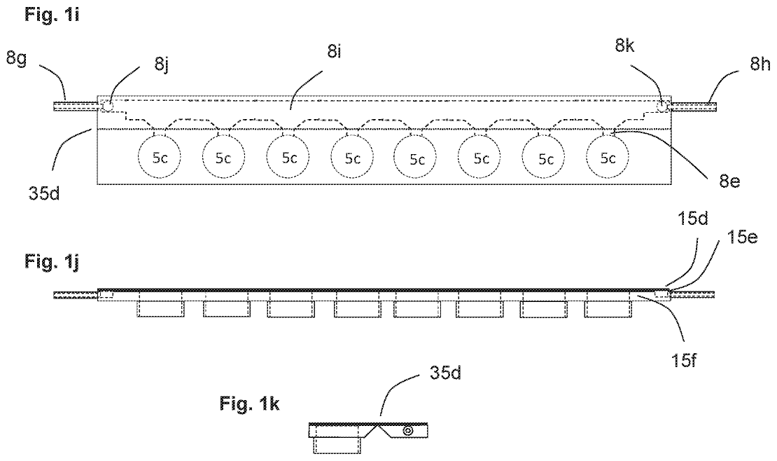

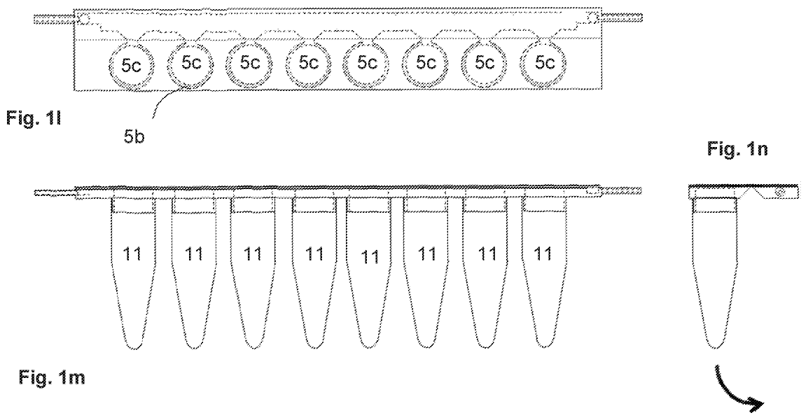

FIG. 1 illustrates some of the features of the liquid distribution device. FIG. 1a is a top (or plan) view and FIG. 1n is a side view. The device has multiple interface ports, in this case one port 21a for the primary PCR tube and eight vents 5 for the secondary PCR tubes. The device is made from multiple layers of material 15a and 15b that can be adhered in patterns that create passageways and bladders. In this case the passageways and the bladders are formed from the same two layers of material. It is also possible to fold a single layer of material onto itself to create two layers. The device shown contains three bladders, input reservoir 1, input reservoir 2, and output reservoir 3. The volume of the bladders is variable from zero to an upper limit determined by the perimeter of the bladder and how much the material creating the walls of the bladder can stretch as shown by the dashed lines in the side view. The device also contains passageways connecting the ports and bladders. The passageways include the passageway 8a that connects the inlet port to the input reservoir 1 bladder, passageway 8b that connects the inlet port to the input reservoir 2, and the zig-zag shaped passageway 8c through 8f that connects the input reservoir 2 to the output reservoir 3.

FIGS. 1c through 1p illustrate alternative embodiments of the fluid distribution portion of the device, in these cases, for interfacing with a strip of eight output sample receptacles 11. In these alternative embodiments, the passageways are formed from different layers of material as are the reservoirs. However, these embodiments could also be manufactured by placing the passageways and reservoirs in the same layers of material.

FIG. 1c shows a top view of the ports that allow fluid to enter 8g and exit 8h the device, fluid to enter 8j and exit 8k the internal chamber 8i, and the vents 5c that allow passage of fluid into the sample receptacles 11.

FIG. 1d shows a side view illustrating examples of the device comprised of two thin layers 15d and 15e that create the upper and lower walls of the chamber 8i, a rupturable membrane layer 15g present in some embodiments of the device, and one thicker layer 15f that contains the fluid vents 5c and structures 5b that interface with the receptacles 11.

FIG. 1e shows samples tubes 11 interfacing with the device.

FIG. 1f (side view) indicates the layers of these examples of the device.

FIG. 1g (top view) indicates the layers of these examples of the device.

FIG. 1h indicates how the layers of these examples of the device are assembled. Two thin layers 15d and 15e are thermally bonded along the dashed lines creating the chamber 8i. This assembly is then bonded to layer 15f. On some embodiments, a rupturable membrane 15g is bonded between the 15d and 15e assembly and layer 15f. If an adhesive is used for bonding, the adhesive does not extend into the areas comprising the vents 5c, and the ports 8j and 8k such that fluid flow is not blocked by the adhesive.

FIGS. 1i through 1p illustrate an example of the fluid distribution portion of the device for interfacing with a strip of eight output sample vials.

FIG. 1i shows a top view of the ports that allow fluid to enter 8g and exit 8h the device, fluid to enter 8j and exit 8k the internal chamber 8i, vents 5c that allow passage of fluid into the sample vials, and vent passages 8e from the internal chamber to the vents 5c.

FIG. 1j shows a side view illustrating one example of the device comprised of two thin layers 15d and 15e that create the upper and lower walls of the chamber 8i, and one thicker layer 15f.

FIG. 1k is a side view rotated 90° relative to the side view in FIG. 1J. The hinging point 35d is shown, which controls fluid flow through 8e, from the chamber 8i into the vents 5c and then into the sample receptacles 11.

FIGS. 1l (top view), 1m (side view), and 1n (side view rotated 90°) show the sample tubes 11 interfaced with the device. The liquid vents 5c and outer structures 5b that interface with the sample tubes 11 are indicated.

FIGS. 1o (top view) and 1p (side view rotated 90°) illustrate the device after the device is bent at the hinging point 35d blocking liquid passage through vent passage 8e.

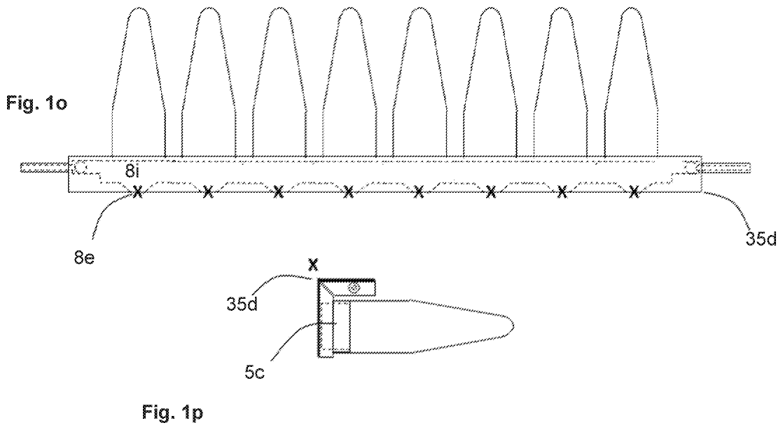

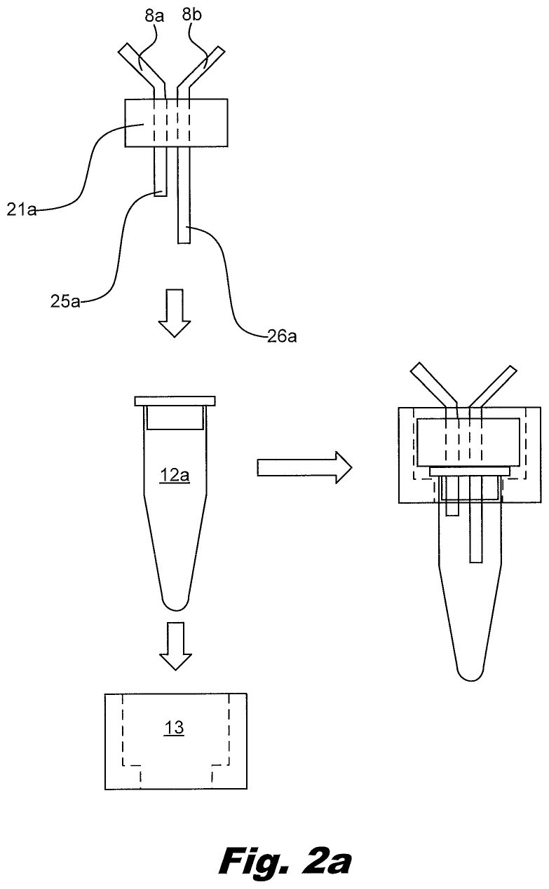

FIG. 2 shows how the primary PCR tube, also termed the transfer vessel, may be attached to the port 21a on the device, in this case the device shown in FIG. 1a. In this example, a sleeve 13 goes over the primary PCR tube 12a. The combination of sleeve 13 and tube 12a engage the port 21a. In this process, two needles 25a and 26a pierce the top of the PCR tube creating a continuous liquid path from the input reservoir 1 through the PCR tube 12a and the input reservoir 2. The sleeve 13 may be threaded to interact with the port 21a. The sleeve 13 may include a keyed feature which interacts with the port. Rotation of the sleeve 13 relative to the tube 12a allows a good seal between the tube 12a and the port 21a without bending and/or damaging the needles 25a, 26a passing through the top of the tube 12a.

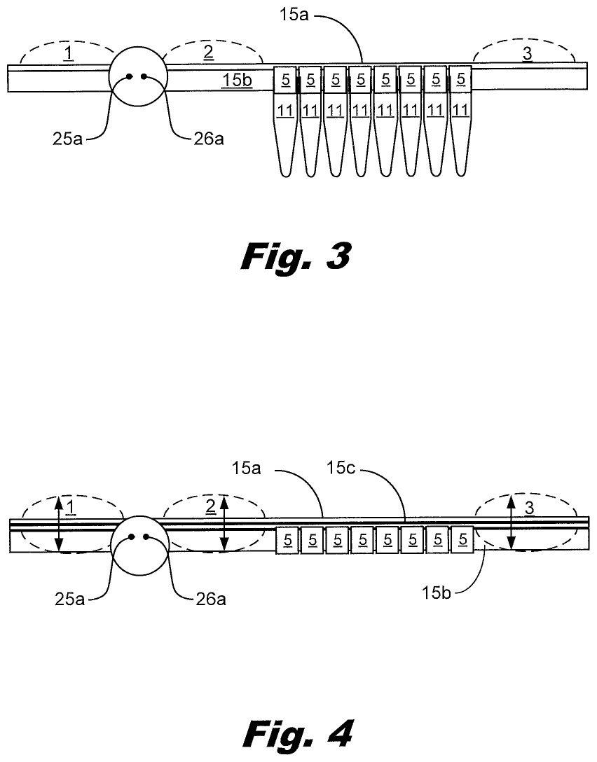

The device shown in FIG. 3 includes a flexible upper layer 15a and a rigid lower layer 15b. FIG. 4 shows an example which has two thin flexible layers 15a and 15c along with a more rigid layer 15b. In some examples, layer 15b has holes and/or passages which allows layer 15c to expand outward in the opposite direction as layer 15a. The passageways and bladders are created by attaching the thin layers 15a and 15c in the pattern of the bladders and flow passageways. The layers 15 may be stock sheet material, individually molded, and/or shaped pieces of polymer. The molding process can be used to produce the components with high reproducibility allowing reproducibility of the mixing and distribution. The top 15a and bottom 15b layers may be made of different materials and/or have different thicknesses. For example, the top portion over the bladders may have a reduced thickness to reduce the force to fill and empty the bladder. The top layer 15a and the bottom layer 15b may be selectively adhered to each other. This may be performed with adhesive, melting portions of a layer, and/or other methods.

The layers 15a, 15b may include features to facilitate alignment and adhesion of the layers. For example, the layers 15a, 15b may include snap fit elements, ridges and groves, extra material for heat welding, etc. In an example, the layers 15a, 15b include a temporary alignment element to hold the layers in position during a secondary adhesion operation. The top layer 15a may be made of a material with a lower melting point and/or lower flow temperature than the bottom layer. For example, the bottom layer 15b may be made from polyurethane and the top layer 15a from a polyethylene. The top layer 15a may be selective over portion of the bottom layer 15b, for example, just covering the bladders and the flow passageways, allowing access to the lower layer 15b. The top layer 15a may include areas of non-uniform thickness. For example, the bladders 1, 2, 3 may be reduced thickness and the gates 35 may have a greater thickness to facilitate blocking of the flow passageway 8. The gates 35 may include larger areas of greater thickness to provide mechanical pressure on the gate 35 and/or allow greater regulation of the gate 35, including intermediate states between open and closed. The layers 15 may be injection molded, heat-formed, and/or created using other techniques depending on the production costs and run size.

FIG. 1a shows an example of a passageway 8 from the input reservoir 2. The passageway 8c exits the input reservoir 2 and includes multiple zig-zags between a first side and a second side. The zig zags form a set of points 8d on the first side. The zig zags form a set of points 8e on the second side. Extending from the second set of points 8e are passageways to the output vents 5.

The passageway 8 receives mixed sample from the input reservoir 2. The mixed sample flows through the passageway 8 and into the output reservoir 3. The mixed sample may flow an amount of sample into the output reservoir 3 to reduce composition gradients in the passageway 8 that may occur during wetting out the passageway 8. For example, a component of the mixed sample may deposit on the sides of the passageway and/or be extracted from the sides of the passageway 8 such that the initial volume of mixed sample differs from the bulk composition. By flowing this initial volume into the output reservoir 3, the mixed sample which is provided to the output ports 5 and the receptacles 11 has a more uniform composition. The output reservoir 3 may also accumulate residual trapped gas, providing a space and minimizing the backpressure produced by the trapped gas. The output reservoir 3 can be substantially reduced in size, or even eliminated, by minimizing the effect of wetting and sample loss, and by removal of trapped gas from the passageways prior to filling with fluid.

In an example, the side passageway includes a valve and/or membrane which obstructs flow. The membrane may be a rupturable membrane. In some implementations the rupturable membrane is a foil. In some implementations the rupturable membrane is a polymer layer with scored locations to rupture. The scoring allows control over where the membrane ruptures and may provide better control over the pressure required to rupture the membrane. Rupturing can be accomplished by pushing on the overlying elastic layer. The elastic layer deflects but the more rigid underlying membrane ruptures.

FIG.3 shows a side view of a device with output vials 11 attached to the output vents 5. The upper layer 15a and lower layer 15b are shown in contact with each other. Dashed lines shown the potential profiles of the reservoirs 1, 2, 3. The two needles 25a, 26a of the port are shown.

FIG. 4 shows a side view of a device for allocating liquid. The output vents to connect to the output receptacle tubes are visible between the input reservoir 2 and the output reservoir 3. A third layer 15c is shown between the upper layer 15a and lower layer 15b. The third layer 15c allows the bladders to distend into recesses in the lower layer 15b in addition to above the lower layer 15b. Dashed lines shown the potential profiles of the bladders 1, 2, 3. The two needles 25a, 26a of the port are shown.

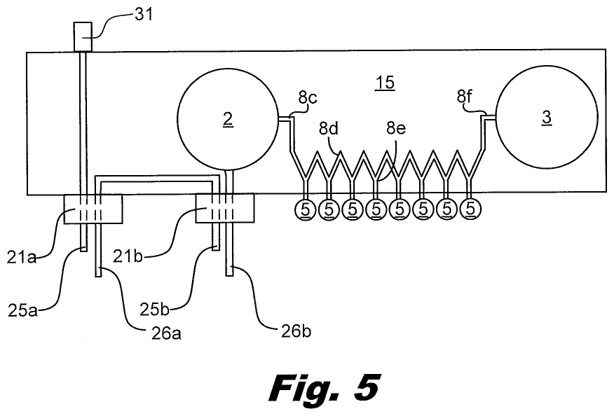

FIG. 5 shows a device for allocating liquid according to one example consistent with this specification. This device does not use an input reservoir 1. Instead a second port 31 is provided. The second port 31 may include a luer fitting and/or similar component to facilitate attachment of a syringe. The second port 31 is used to provide the dilutant. The second port also provides pressure to move liquid through the device. This may facilitate control over pressing on the input reservoir 1. This may also allow integration with automated and/or semi-automated volume controls.

FIG. 5 also includes two ports to attach vials 21a and 21b. This allows mixing two different samples into the mixed solution provided to the output vials. In an example, a single port 21b is connected to the second port 31. In an example, additional ports 21, for example, three, four, five, and/or more, may be added to allow more samples to be combined without exposure to the environment.

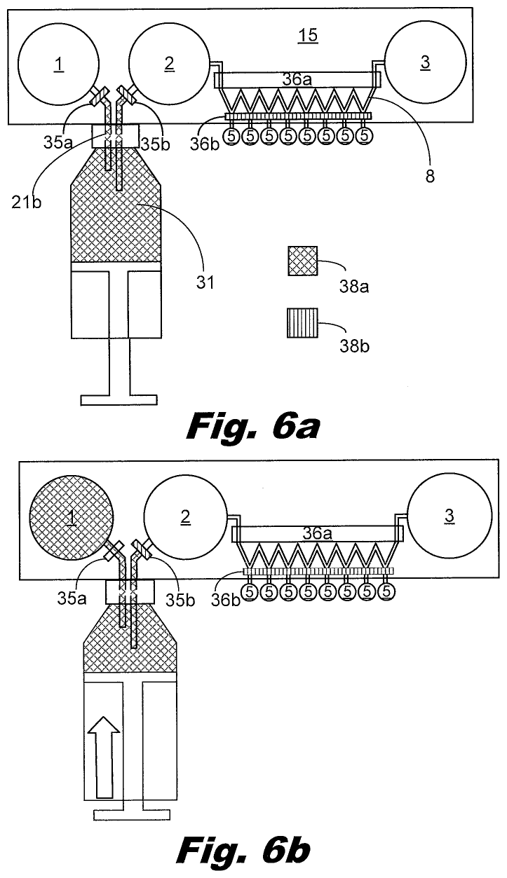

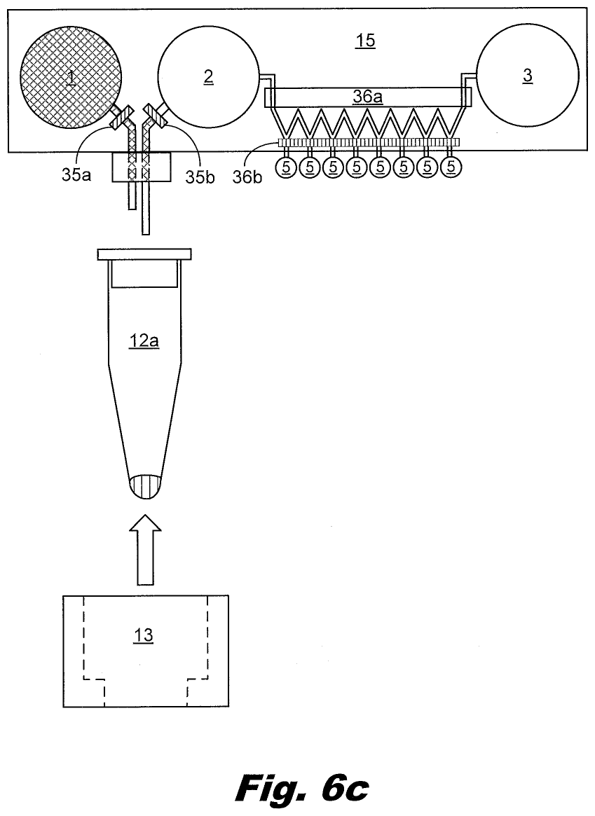

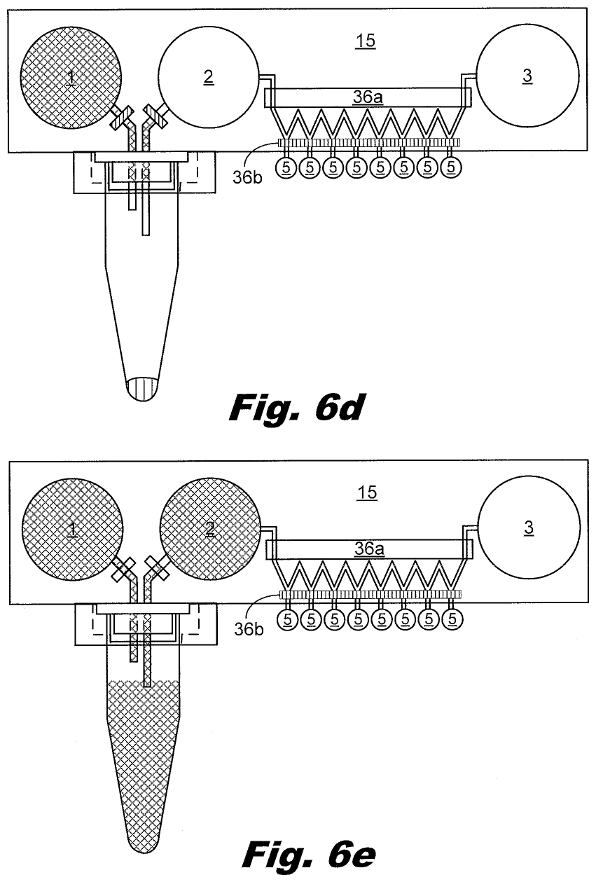

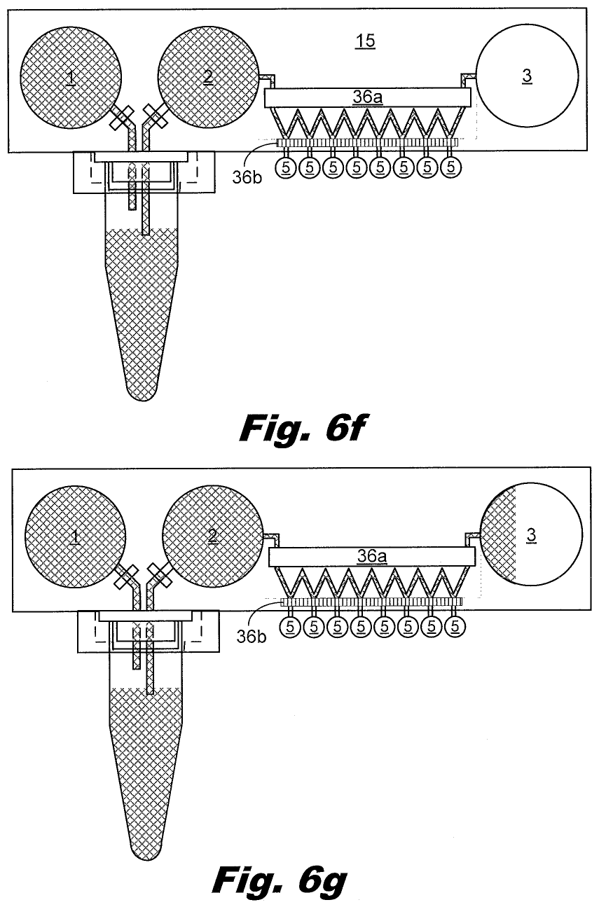

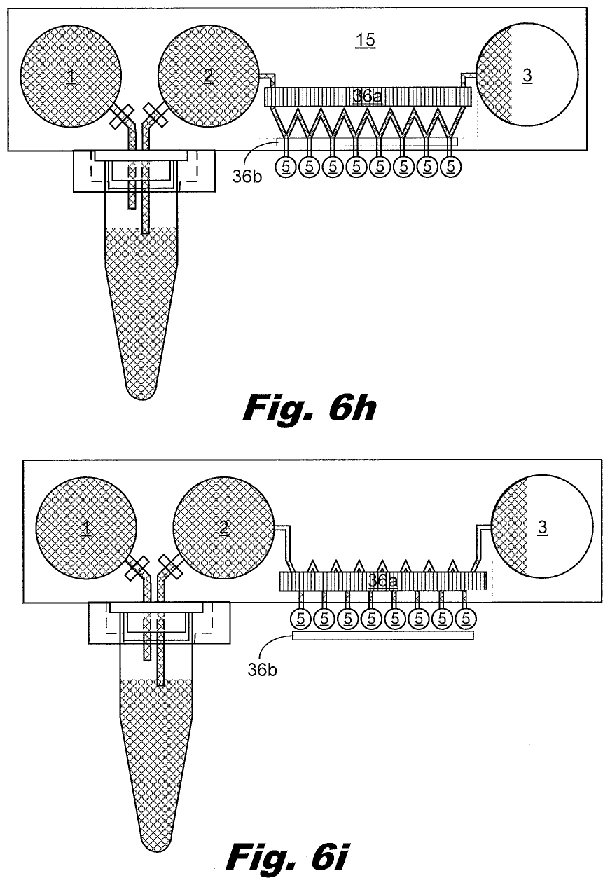

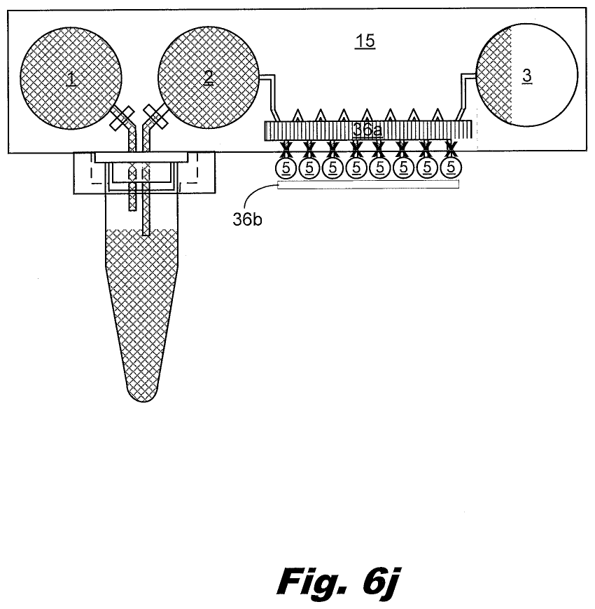

FIGS. 6a-6j illustrates the operation of the device. The force to move liquid is generated by applying force to compress the input reservoir bladders 1 and 2. Flow force may be provided by the syringe 31, and/or rollers 36a, 36b. Scrapers may be used in the place of rollers 36. Liquid flow is regulated by applying force to locations on the passageways 8 using gates 35a, 35b in order to squeeze the orifice of the passageway shut and block flow, and/or by rollers 36 to move the liquid within the passageway using a peristaltic effect. Liquid is indicated by a crossed hatching 38a. An open passageway is indicated using unhatched element, such as 36a. A closed passageway is indicated using lined hatching 38b.

FIG. 6a illustrates the first step in the process. The passageways 8a and 8b are closed by applying force at gates 35a and 35b and a syringe 31 prefilled with liquid 38a is attached to the primary PCR tube port 21b. When the bladders, passageways, and syringe contain liquid, the liquid is indicated by crosshatch 38a.

FIG. 6b illustrates the second step in the process. Pressure is relieved at gate 35a, the plunger on the syringe 31 is depressed, and liquid flows through the passageway 8a into the input reservoir 1.

FIG. 6c illustrates the third step in the process. Pressure is applied to gate 35a to block the passageway 8a. The syringe 31 is removed. The primary PCR tube 12a is placed inside the sleeve 13, and then pressed upwards against the needles 25a and 25b to puncture the cap of the tube 12a. The sleeve 13 is engaged with the port 21a.

FIG. 6d shows the primary PCR tube 12a in place on the device.

FIG. 6e illustrates the fourth step in the process. Pressure is relieved at gates 35a and 35b and pressure is applied to the input reservoir 1. This causes liquid to flow through the passageway 8a, through the primary PCR tube 13a mixing with the primary PCR products and carrying these products out of the tube, through the passageway 8b and into the input reservoir 2. Once the liquid is in the input reservoir bladder 2, passageway 8b is blocked at gate 35a to prevent backflow. In another example, a one way valve in passageway 8b may prevent backflow.

FIG. 6f shows the mixed liquid advancing from the input reservoir 2 through the passageway 8 to the output reservoir 3. The scraper and/or roller 36b are engaged, preventing the liquid from flowing into the outflow passageways to the output vents 5 and into the output receptacles 11.

FIG. 6g shows the mixed liquid advancing into the output reservoir 3 which is partially filled. Enough liquid is flowed through into the output reservoir 3 to provide a uniform concentration through the passageway 8 between the input reservoir 2 and the output reservoir 3. Pressure applied to the input reservoir 2 moves the liquid through the passageway 8 and into the output reservoir 3.

FIG. 6h illustrates the fifth step in the process. Downward force is placed on the roller 36a blocking the passageways 8c and 8f. Then both rollers 36a and 36b are rolled toward the output vents 5. In an example, the roller 36b is removed off the top surface of the upper sheet 15a. This avoids the need to move both rollers 36a and 36b. In another example, a one way valve may replace roller 36b.

FIG. 6i illustrates the sixth step in the process. As the roller 36a is moved toward the output vents 5, liquid in the passageway 8 is moved into the side passageways connected to the output vents 5. Eventually, the roller 36a causes liquid to be pushed from the passageways 8 into the output vents 5 and aliquot receptacles 11.

FIG. 6j illustrates the seventh step in the process. The aliquot receptacles 11 are sealed using pressure and heat, then cleaved using heat and/or pressure applied at the locations indicated by the “X”s. The aliquot receptacles 11 may then be centrifuged to bring the aliquoted liquid to the bottom, such that the aliquoted liquid is ready for PCR cycling. The remainder of the device 15 may be discarded. The aliquot receptacles 11 may be PCR tubes, which are compatible with other existing lab equipment.

The disclosed operation of the device includes gates 35, rollers 36, and similar mechanical elements for regulating action in the device. These elements may be manually controlled, automated, and/or semi-automated. In an example, the system is attached to a dilutant (solution for dilution) source, a sample to dilute, and the output containers before executing an automated protocol. In another example the system is loaded with dilutant, for example with a syringe via a luer fitting, and then attached to a vial containing the material to be diluted.

Access to the transfer vessel vial may be made through an open top. The risk of contamination may be reduced by puncturing a portion of the vial to minimize exchange between the environment and the sample to be diluted. In an example, the output vents 5 for accessing the vials 11 extend below the device. For example, there may be a mounting block to hold the sample vial in place under the device. The mounting block may also hold aliquot receptacles 11 to receive the diluted aliquots.

In an example, the gates 35, rollers 36, scrapers and mechanical elements are static in X and Y, moving only in the vertical axis. The device is on a plate with at least one axis of motion, for example Y. This may allow integration with existing motion plates and robotics for liquid handling. The gates 35 and rollers 36 may also be capable of motion in two or more axes. For example, the rollers may be capable of both vertical motion to engage with the system and lateral motion to express the liquid from the device into the aliquot receptacles 11.

The outer roller 36b (toward the output vials) may be a valve/pressor that mechanically seals the connection to the aliquot receptacles. If so, the outer pressor 36b may have a smaller width compared with the roller to minimize interaction between the roller and the bottom pressor during expression of the liquid.

The system may include a substrate block with holes allowing light based assessment of the output aliquot receptacles. The system may include elements to thermally cycle the output aliquot receptacles to perform a secondary amplification. The system may include elements to perform the primary amplification on the sample source.

Another variation uses two passageways 8 from the input reservoir 2. The first passageway 8 functions as describe above. The second passageway functions similarly but is oriented toward an opposite side of the device with a second group of output vents 5. This allows a sample to be diluted to two sets of secondary PCR tubes instead of a single set. A single roller 36a may provide both press/roll operations. The device may use different rollers 36a for each set of secondary PCR tubes. The two passageways 8 may connect to a common output reservoir 3 or the two passageways 8 may use separate output reservoirs 3. The two passageways may have similar geometries or may have different geometries to allow for a wider variety of sample sizes.

An additional layer may be applied above the upper layer 15a and/or below the lower layer 15b. This additional layer may include mechanical and/or hydraulic and/or pneumatic elements to close the passageway 8 between the upper layer 15a and lower layer 15b.

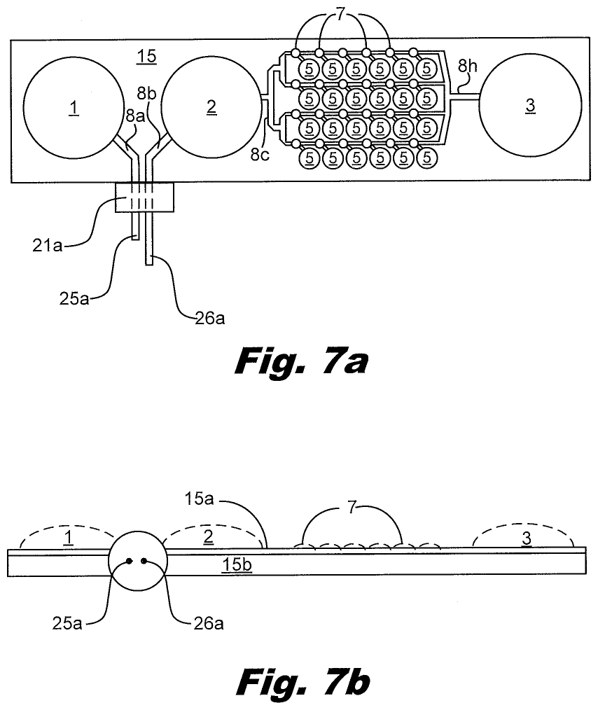

FIGS. 7a and 7b show a system to perform the secondary PCR reaction in plates of PCR wells rather than individual tubes or strips of tubes. FIG. 7a is a top (or plan) view and FIG. 7b is a side view. In this example, the zig-zag passageways 8 and rollers are replaced with bifurcating passageways 8c, multiple parallel passageways 8i, and a series of aliquot bladders 7 located along the path the parallel passageways. The vents 5 are arranged in an array to match the PCR plate. In this case a four by six array of vents is shown, but the system may be arranged as a 4 by 8 array, a 6 by 8 array, an 8 by 12 array, or other array patterns as well. The vents 5 could be associated with multiples types of structures in order to interface with the aliquot receptacles. These structures could include a circular lip that allows them to snap into the aliquot receptacle tubes 11 or a layer of adhesive which bonds to the aliquot receptacle tubes.

In an example, the branching passageways 8 include narrow portions which reduce the passageway 8 to passageway 8 variations. Other approaches can be used to distribute the liquid to the passageways 8 and the respective bladders 7. For example, a manifold may be used between the input reservoir 2 and the passageways 8. A manifold can be used to control the order in which passageways 8 fill. Good design in this respect can avoid trapping air in the passageways. In an example, the input reservoir 2 includes multiple outputs which feed different passageways 8 with their respective aliquot bladders 7. In an example, the outputs of the passageways 8 do not consolidate but individually feed into separate output reservoirs 3. In this manner, all passageways can be filled independent of whether the passageways fill simultaneously or sequentially.

In an example, a rigid, lower second layer 15b may include features to interface with a well plate. For example, the lower surface of the rigid lower layer 15b may include protrusions, guides, recesses, and/or similar mechanical features to position and/or retain the rigid lower layer 15b on the well plate. In an example, the output vents 5 include features to center them in the wells of the well plates. The rigid lower layer 15b may seal the wells of the well plate to reduce the possibility of contamination.

A second lower layer 15b may support a flexible lower layer 15d which seals the wells of the well plate. In an example, once the liquid is distributed to the wells, the distribution passageways are sealed, isolating the wells. The support layer 15a and/or the rigid support layer 15b may be separated from the layer and/or layers used to seal the wells. In an example, the lower part of the device includes an adhesive which attaches the device (temporarily or permanently) to the top of the well plate.

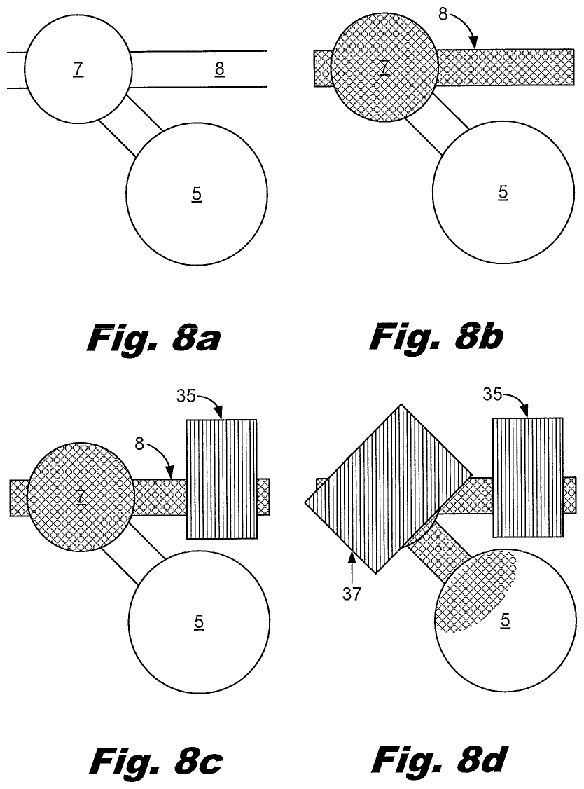

FIGS. 8a-d illustrates the operation of the array interface device. Filling the reservoir bladder, interfacing with the primary PCR tube, and filling the input reservoir are the same operations as shown in FIG. 6a through 6e and therefore not illustrated.

FIG. 8a shows the portion of the system prior to filing with the mixed liquid from the input reservoir 2. In FIG. 8b, the passageways 8 and integral aliquot bladders 7 are filled with the liquid to be distributed to the wells. In FIG. 8c, the volumes with the aliquot bladders 7 are isolated from each other. In FIG. 8d, a plunger 37 presses downward on the isolated aliquot bladder 7. The plunger 37 forces the liquid into the connecting vent passageway and into the PCR tube 5 and/or a well in the well plate.

The force to move liquid is generated by applying force to compress the bladders 1, 2, 3, and 7. However, pressers 37 activated vertically (from above and/or below) may be used in the place of rollers 36b or scrapers 36a. Liquid flow may be regulated by applying force to locations on the passageways 8 using gate 35 to block the passageway 8 shut and block flow, or by the pressers 37 to move the liquid within the aliquot bladders 7 into the aliquot receptacles 11. Liquid is indicated by a crossed hatching 38a. An open passageway is indicated using unhatched element, such as 36a. A closed passageway is indicated using lined hatching 38b.

After the liquid has been expressed, the passageways 8 connecting the aliquot bladders 7 and the aliquot receptacles 11 and/or wells may be sealed using pressure and/or heat, then cleaved using heat and/or pressure. The secondary PCR plate and/or PCR tubes 5 may be centrifuged to bring the aliquoted liquid to the bottom of the well to prepare the plate and aliquoted liquid for PCR cycling. The remainder of the device may be discarded.

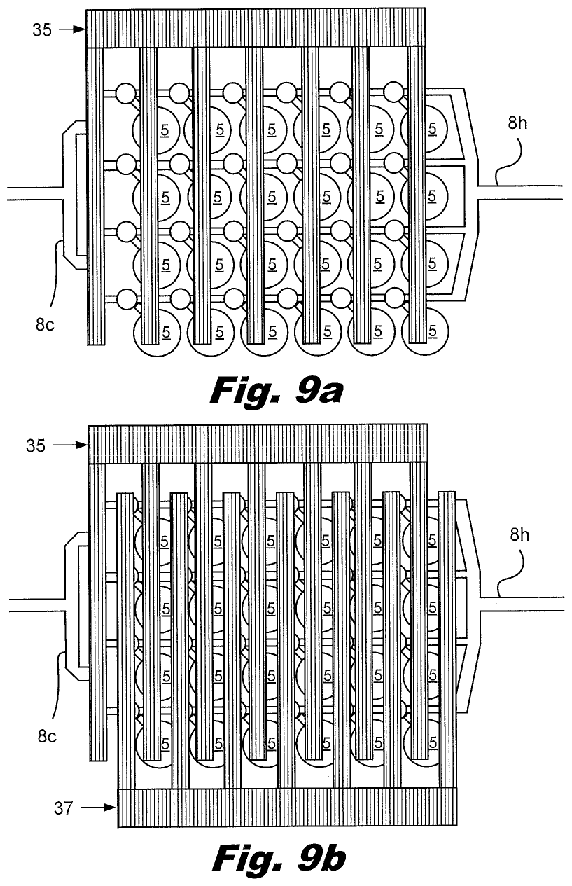

FIGS. 9a and 9b show a system view of the operations of isolating the aliquot bladders 7 and expressing their contents into the wells and/or the aliquot receptacles 11. In FIG. 9a, a presser 35 is used to isolate multiple aliquot bladders 7 from each other. In FIG. 9b, a plunger 37 is used to compress the aliquot bladders 7 and express the liquid into the wells, aliquot receptacles 11 and/or PCR tubes. A wide variety of mechanical systems can be implemented to perform the operations of isolating and expressing the aliquot bladders 7 into the well of a well plate and/or the aliquot receptacles 11. For example, heated pressor 35 may also seal the passageways 8 between the aliquot bladders 7 before the plunger forces the liquid into the aliquot receptacles 11. The passageways 8 may be sealed and cleaved using pressure and/or heat. In an example the passageways 8 are sealed at the exit of the input reservoir 2 and the entrance to the output reservoir 3. This may facilitate handling and/or storage of the mixed liquid prior performing the secondary PCR. The passageways between the aliquot bladders 7 and the aliquot receptacles 11 and/or wells may be sealed using heat and/or pressure, and cleaved using heat or a mechanical cutter such as a blade. Residual nucleic acids at the cleaved surfaces can be eliminated using heat, chemical methods such a bleach treatment, or irradiation such as UV irradiation. Eliminating these nucleic acids reduces the opportunities for contamination.

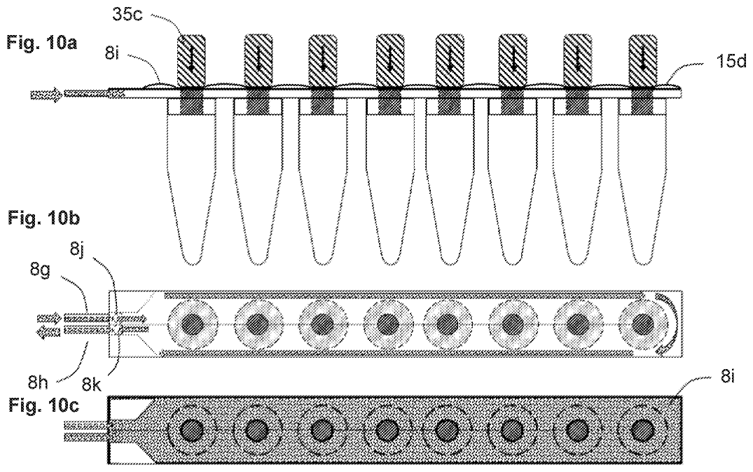

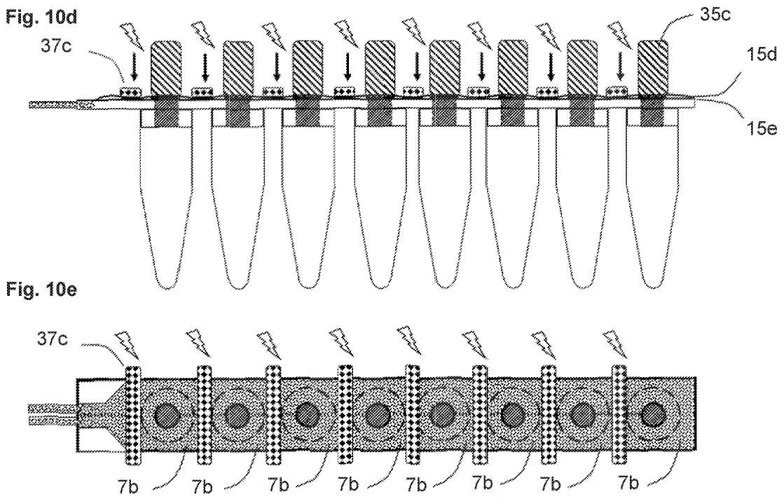

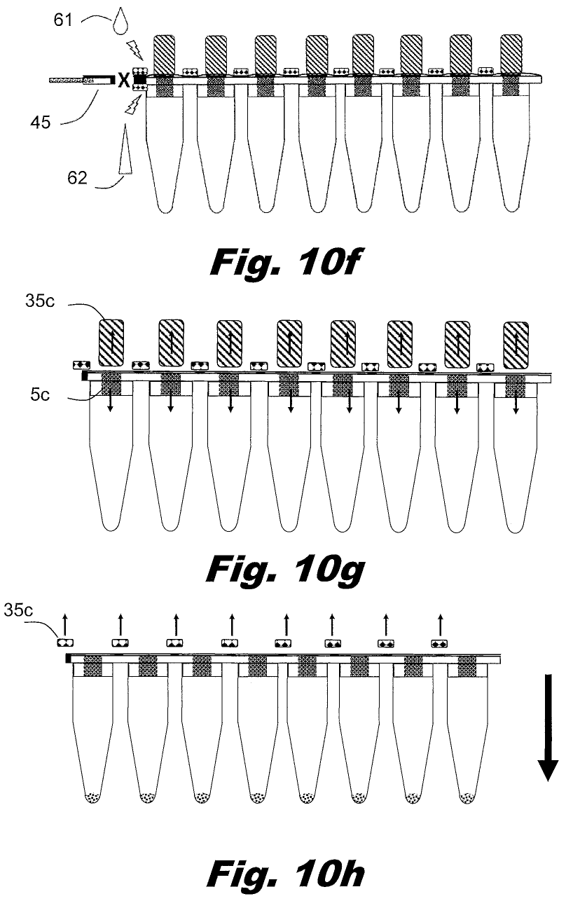

FIGS. 10a through 10h illustrate function of the device shown in FIGS. 1c through 1h.

FIG. 10a is a side view of the device. Pressers 35c press the thin layers 15d and 15e against the vents 5c thereby blocking the passage from the chamber 8i into the aliquot receptacle vials.

FIGS. 10b and 10c are top views of the device. FIG. 10b indicates how liquid can flow into the chamber 8i via the ports 8g and 8j, past the closed vents 5c, around the end of the chamber and back in the opposite direction, then back out of the chamber through ports 8k and 8h.

In FIG. 10c, the outlet vent 8h is then blocked. Liquid subsequently forced with modest pressure into the inlet port 8g can expand the upper surface of the chamber (indicated 15d in FIG. 10a). The degree of this expansion is dependent on the elasticity and thickness of the chamber walls and the pressure applied.

FIGS. 10d (side view) and 10e (side view) indicate sealing bars 37c contacting the device between the pressers 35c, displacing the liquid within the chamber from beneath bars. Electrical current is then passed through the sealing bars heating the sealing bars and thermally welding the two layers of the chamber 15d and 15e. This action creates multiple individual smaller chambers 7b containing aliquots of liquid.

FIG. 10f (side view) indicates sealing, cleavage, and removal of the portion of the fluidic device 45 that is no longer needed and would otherwise interfere with downstream operations and/or instruments, such as a centrifuge and/or a qPCR machine. Sealing can be accomplished using approaches such as heating and/or adhesives. Cleavage may be accomplished using multiple mechanisms such as heating above the melting temperature, a mechanical blade 62, and/or laser cutting. To prevent the release of materials such as PCR products at the cleaved interface, the heating can be carried out at elevated temperatures and extended times such that the PCR products at the cleavage site are chemically degraded. Alternately, and/or in addition to heat, a chemical agent, such as bleach 61, may be applied to degrade the nucleic acid sequences. Ionizing and/or non-ionize radiation may be applied to degrade the nucleic acid sequences.

FIG. 10g (side view) indicates release of the pressure applied by the Pressers 35c thereby opening the passages through the vents 5c, which allows the aliquots of liquid in the small chambers 7b created by the sealing bars to flow into the aliquot receptacles 11.

FIG. 10h (side view) indicates release of the pressure from the sealing bars 37c and subsequent centrifugation to force all the liquid that entered the aliquot receptacle 11 to the bottom of the aliquot receptacle 11 for more efficient and reliable downstream processing.

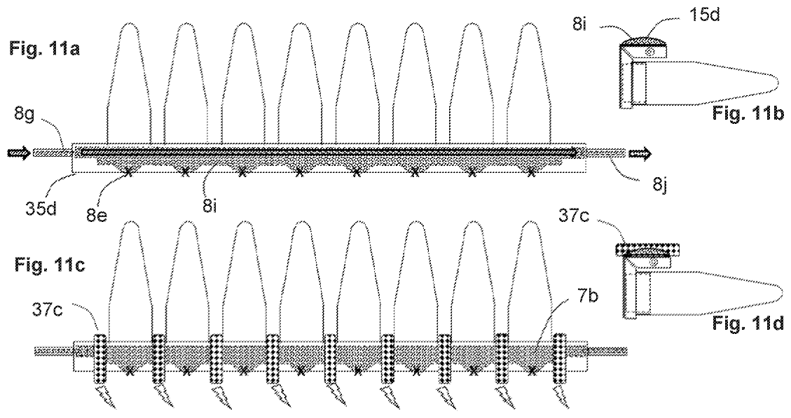

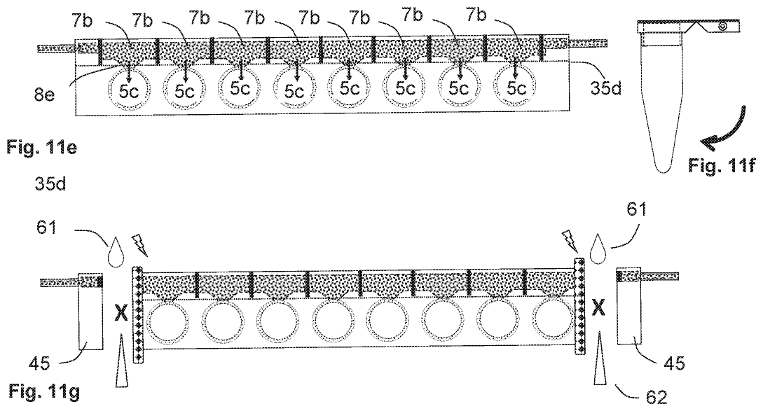

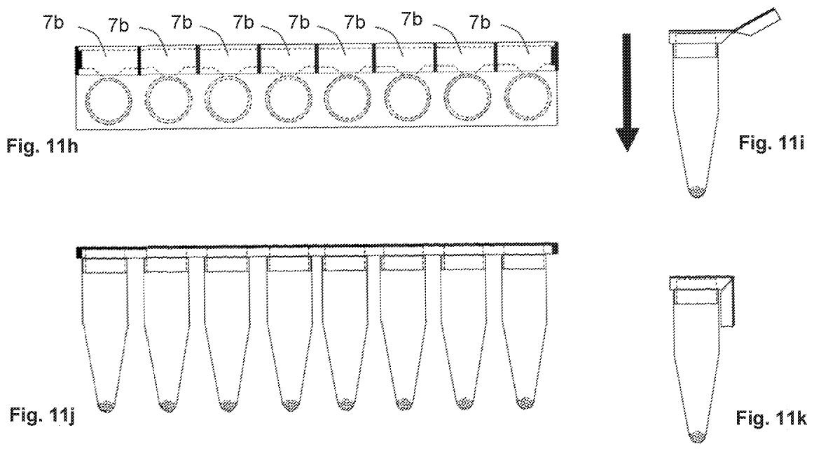

FIGS. 11a through 11k illustrate function of the device shown in FIG. 1i through 1p.

FIG. 11a is a top view and 11b is a side view of the device that indicates bending at the hinging point 35d to close the passages 8e. Then liquid is forced into port 8g filling the chamber 8i by expanding the elastic top layer 15d. The liquid flows past the closed passages 8e, and out of the chamber through port 8j. Port 8j is then blocked and the volume in the chamber 8i is dependent on the elasticity and thickness of the chamber walls 15d and 15e and the pressure applied.

FIGS. 11c (side view) and 11d (side view) indicate the sealing bars 37c contacting and pressing against the surface of the device at regular intervals thereby displacing the liquid within the chamber from beneath bars, then thermally welding the two layers of the chamber and creating multiple individual smaller chambers 7b containing aliquots of liquid.

FIG. 11e (top view) and 11f (side view rotated 90°) indicates unbending at the hinge point 35d thereby opening of the passages 8e allowing flow of liquid from the aliquots 7b through the vents 5c and into the aliquot receptacle vials.

FIG. 11g (top view) indicates sealing, cleavage, and removal of the portions of the fluidic device 45 that are no longer needed. These portions may interfere with subsequent sample handling operations. The sealing and cleavage can be accomplished using multiple mechanisms such as heating past melting temperature, a mechanical blade 62, and/or laser cutting. To prevent PCR product contamination release at the cleaved interface, the heating can be carried out at elevated temperatures and extended times, and/or by using a chemical agent such as bleach, such that the PCR products at the cleaved interface are chemically degraded and/or irradiation such as UV irradiation

FIGS. 11h (top view), 11j (side view) and 11i (side view rotated 90°) indicate the device can now be centrifuged to force the liquid from the aliquots 7b to the bottom of the aliquot vials for more efficient and reliable downstream processing. FIG. 11i indicates the hinge point can be bent upward slightly for more efficient evacuation of liquid from the aliquots into the bottom of the aliquot vials during centrifugation.

FIG. 11k indicates the hinge point is now bent down to allow for the proper tube spacing for multiple devices used in downstream sample processing.

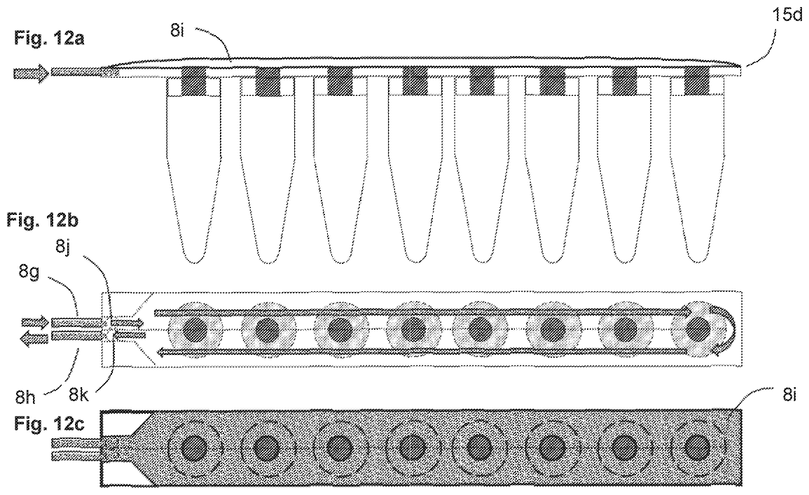

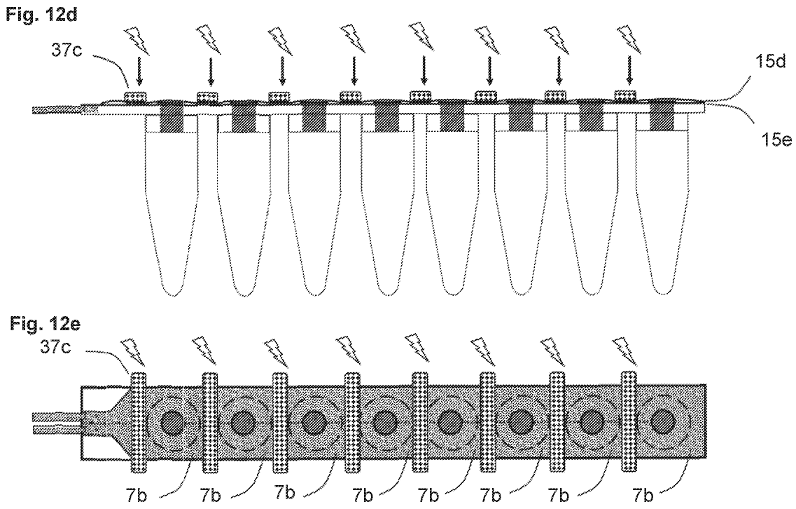

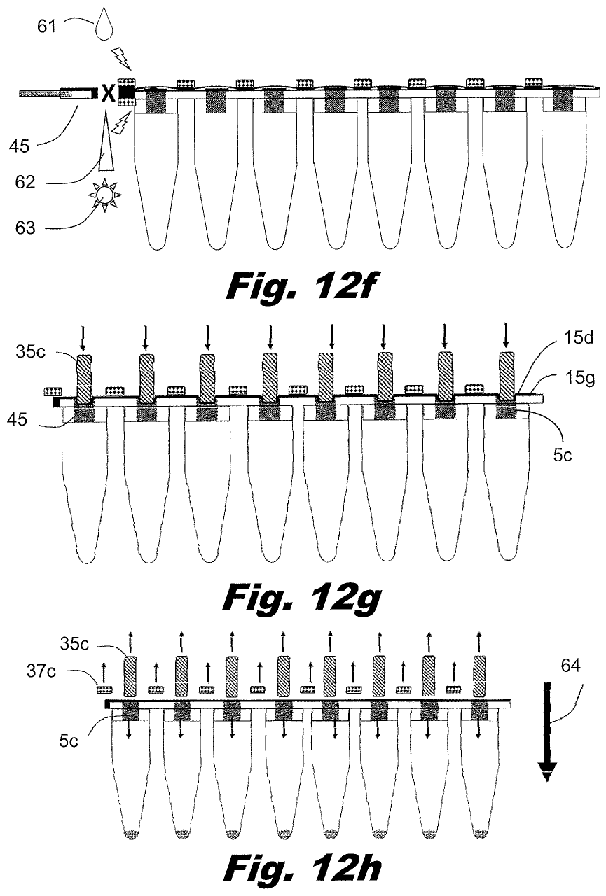

FIGS. 12a through 12h illustrate function of the device shown in FIGS. 1c-through 1h that contains the rupturable membrane.

FIG. 12a is a side view of the device. A rupturable membrane 15g blocks the passage of liquid from the chamber 8i into the aliquot receptacle vials.

FIGS. 12b and 12c are top views of the device. FIG. 12b indicates how fluid can flow into the chamber 8i via the ports 8g and 8j, over the vents 5c that are blocked by a rupturable membrane, around the end of the chamber and back in the opposite direction, then back out of the chamber through ports 8k and 8h.

In FIG. 12c, the outlet port 8h is then blocked and fluid forced into the inlet port 8g with modest pressure, the upper surface of the chamber (indicated as 15d in FIG. 12a) can expand as it is an elastic layer of material. The degree of this expansion is dependent on the elasticity and thickness of the chamber walls and the pressure applied.

FIGS. 12d (side view) and 12e (top view) indicate sealing bars 37c contacting the device between the pressers 35c, displacing the liquid within the chamber from beneath bars. Electrical current is then passed through the sealing bars heating the sealing bars and thermally welding the two layers of the chamber 15d and 15e. This action creates multiple individual smaller chambers 7b containing aliquots of fluid.

FIG. 12f (side view) indicates sealing, cleavage, and removal of the portion of the fluidic device 45 that is no longer needed and would otherwise interfere with downstream operations and/or instruments, such as a centrifuge and/or a qPCR machine. The sealing and cleavage may be accomplished using multiple mechanisms such as heating, a mechanical blade 62, and/or laser cutting. To prevent the release of materials such as PCR products at the cleaved interface, the heating can be carried out at elevated temperatures and extended times such that the PCR products at the cleavage site are chemically degraded. Alternately, and/or in addition to heat, a chemical agent, such as bleach 61, or UV irradiation 63 may be applied to degrade the nucleic acid sequences.

FIG. 12g (side view) indicates rupturing of the membrane 15g. Downward pressure is applied by the pressers 35c on top of the layer 15d. Because layer 15d is substantially more elastic that the rupturable membrane 15g, the elastic layer flexes whereas the membrane below cannot flex the same distance and therefore ruptures thereby opening the passages through the vents 5c, which allows the aliquots of fluid to flow into the sample receptacle vials. In some examples, the rupturing of the membrane is accompanied by a mechanical element. In an example implementation, the rupturable membrane is a metal foil. In another implementation, the rupturable membrane is a scored polymer membrane.

FIG. 12h (side view) indicates release of the pressure from the pressers 35c and the sealing bars 37c and subsequent centrifugation 64 to force all the fluid that entered the vents 5c into the bottom of the vials for more efficient and reliable downstream processing.

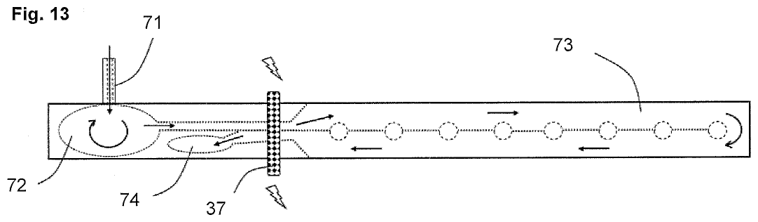

FIG. 13 shows an arrangement demonstrating how the liquid aliquot portion of the device, shown in FIGS. 1c, 1d, and 1e, can be incorporated with the upstream and downstream fluidics including the input reservoir bladder 72 and the transfer vessel. In FIG. 13, there is port 71 to interface with an input sample transfer vessel. Liquid is provided to the mixing bladder 72, and then flows through a passage 73, which acts as the liquid distribution portion of the device, then the first part of the liquid reaches the output reservoir 74. The area 37 shows the sealing and/or cleavage operation that separates the liquid distribution portion from the rest of the device.

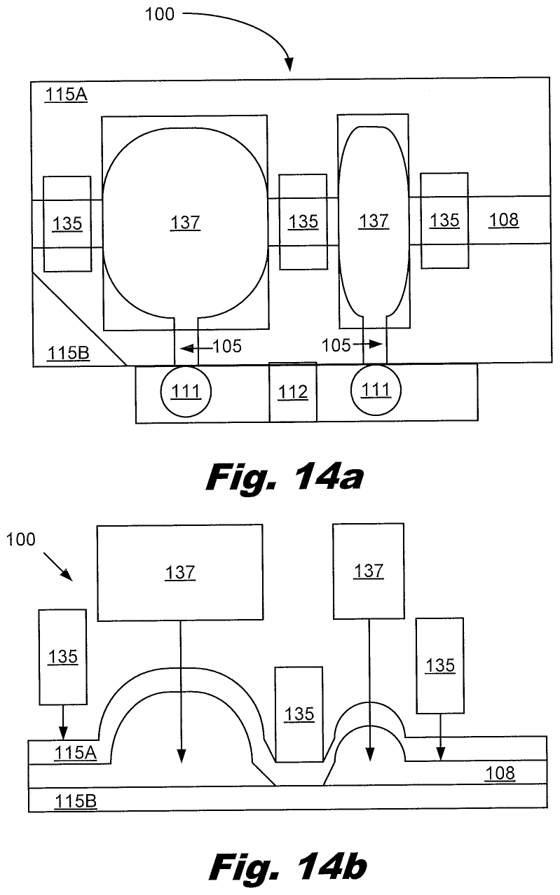

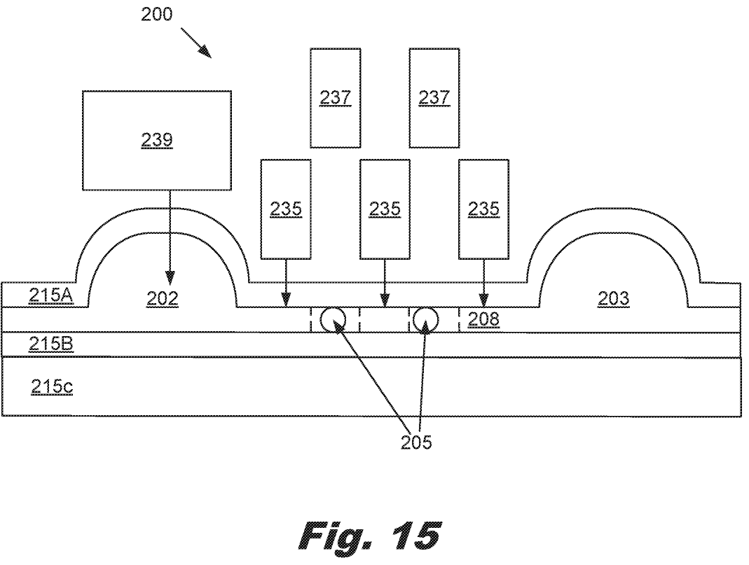

FIG. 14a shows a top view of device 100 for forming a liquid aliquot according an example consistent with this specification. FIG. 14b shows a side view of the same device 100. The device 100 includes: an elastic, first layer 115A; second layer 115B overlapping the first layer 115A; a first passageway 108 to receive and hold a volume of liquid, the first passageway 108 formed from the first 115A and second layers 115B; a first actuator 135 to press on the elastic layer 115A thereby dividing the liquid filled passageway 108 into a series of liquid aliquots; a series of vents 105 associated with the series of aliquots; a second actuator 137 to control flow of liquid aliquots through the associated vents 105; and an attachment structure 112 for attachment of aliquot receptacles 111 to receive liquid aliquots that flow through the vents 105.

The device 100 is a device 100 for forming a liquid aliquot. The device 100 may form multiple aliquots. The aliquots may have the same or different volumes. The device 100 may reduce the incidence of contamination and/or false positives associated with transferring products.

The device 100 includes an elastic first layer 115A that overlays a second layer 115B. The elastic first layer 115A and second layer 115B form the walls of various features used to perform the desired metering of the liquid. The elastic first layer 115A and second layer 115B form a passageway 108 between them. The passageway 108 has an entrance and an exit. The passageway 108 can expand to receive liquid. The elastic first layer 115A and second layer 115B may be formed from polymers. In an example, one or both layers 115 maybe formed from a thermoplastic, allowing remodeling using heat, for example to seal portions of the passageway 108.

The passageway 108 contains no volume when empty but can be filled with fluid due to expansion of one or more of the elastic walls. The passageway may be undulated. The passageway 108 may zig-zag. The passageway 108 may include a plurality of connected chambers. The passageway 108 may be designed to allow isolation of the volumes using a simple mechanical actuation. A variety of suitable geometries are shown in the figures. Each isolate volume of the passageway 108 has an associated vent 105. The vent 105 is used to transfer the liquid from the isolated portion of the passageway 108 into the desired aliquot receptacles 111. Opening and closing of the vent 105 can be controlled.

The volumes of the aliquots may be uniform. The volumes of the aliquots may be of different. The volumes of the aliquots are portions of the passageway 108, which may be isolated by the first actuator 135. The first actuator 135 isolates the portions of the passageway 108 from each other. This prevents communication between the isolated volumes (and the associated liquid aliquots) during transfer of the aliquots to their receptacles 111.

Each aliquot volume has an associated vent 105. The vent 105 may be closed during filling of the passageway 108. When the vent 105 is opened, by using a mechanism such as relieving pressure, unfolding, or rupturing a membrane, liquid from passageway 108 can flow through the vent 105 and into the receptacle 111. The receptacle 111 may be a tube, a vial, a well, and/or other desired container. The first actuator 135 isolates the volumes of the passageway 108. In an example. the first actuator 135 presses down on portions of the passageway 108 to isolate the volumes. The first actuator 135 may include heat and/or pressure. The portions of the passageway 108 closed by the first actuator 135 may include features to minimize the volume in the closed portion. The portions may include mechanical features to create a transition from open to close. In an example, the isolation of the volumes is not reversible. The first actuator 135 may actuate a latch and/or similar mechanism to hold the portions of the passageway 108 closed.

The second actuator 137 may apply pressure to the second sheet over the isolated volume. This pressure causes the liquid in the isolated volume to be transferred through the vent 105 into the aliquot receptacle 111. The second actuator 137 may actuate volumes simultaneously. The second actuator 137 may actuate volumes sequentially. The second actuator 137 may move laterally from one side of the volume to the other to drive the liquid in the isolate volume into the vent. The second actuator 137 may have a slopped contacting portion which contacts the volume away from the vent 105 and gradual presses the elastic first layer 115A down toward the vent 105.

The second actuator 137 may be a rupturable membrane, wherein rupturing of the membrane controls the flow of a liquid aliquot through the associated vent. In this example, the vent 105 is blocked by the rupturable membrane. The pressure on the membrane is increased until it ruptures, then the liquid aliquot is able to flow through the vent 105 to the aliquot receptacle 111.

In an example, the second actuator 137 is a presser, wherein pressing the second elastic layer toward the first layer controls the flow of a liquid aliquot through the associated vent 105. The pressor may obstruct the vent. The vent 105 may open under pressure when the pressor pushes down on the first elastic layer 115A over the aliquot.

The second actuator 137 may be a roller. The roller may force a liquid aliquot through a vent 105 using a peristaltic effect. The roller may push down on the elastic layer 115 from one side of the aliquot and push toward the vent 105.

The device 100 may include an input reservoir 2; the input reservoir 2 may be located between the elastic first layer 115A and second layer 115B and the input reservoir 2 feeding the passageway 108. The entrance to the passageway 108 may be directly connected to the input reservoir 2 similar to the input reservoir 2 described to in FIG. 1. The input reservoir 2 may include an actuator to agitate and/or mix liquid in the input reservoir 2. In an example, a rotating nub presses against the elastic first layer 115A above the input reservoir 2 and agitates the liquid in the input reservoir 2.

The device 100 may include an actionable valve between the input reservoir 2 and the passageway 108. The valve may be closed during mixing in the input reservoir 2. The valve may open once a predetermined pressure is applied to the valve. In an example, the inflow(s) to the input reservoir 2 are blocked and then pressure is applied to the elastic first layer 115A above the input reservoir 2. Once the pressure reaches a predetermined threshold, the valve opens and liquid flows into the passageway 108 expanding the passageway 108 and forcing any trapped air out the end of the passageway 108.

The end of the passageway 108 may connect to an output reservoir 3 similar to the output reservoir 3 described to in FIG. 1. The output reservoir 3 receives any residual air from the passageway 108 and an initial amount of liquid flowed through the passageway 108. In some example, the initial liquid has a different composition from the bulk liquid and flowing the initial liquid into the output reservoir 3 provides more uniform samples in the aliquots. It may also be possible to eliminate the output reservoir 3 if the passageways are completely empty and devoid of air prior to fluid being injected, and if the walls of the passageway to not alter the input fluid.

FIG. 15 shows an example of a system 200 for preparing a liquid aliquot consistent with this specification. The system 200 includes: an elastic passageway 208 connected at one end to an input reservoir 202; a third actuator 239 to flow liquid from the input reservoir 202 into the passageway 208; a first actuator 235 to divide the passageway 208 into a plurality of liquid aliquots; and a second actuator 237 to control the flow of a liquid aliquot from an isolated portion of the passageway 208 through a vent 105 and into an attached aliquot receptacle 111.

The elastic passageway 208 may include an elastic first layer 215A and second layer 215B form a number of different features. The layers 215A, 215B form part of the liquid handing volumes of the device 300. The layers 215A, 215B allow pressure to be applied to liquid volumes on the device 300 by pressing on the upper (outer) surface of the elastic first layer 215A to apply pressure and/or to flow the contained liquid. In some examples, some portions of the passageway 208 are elastic and readily distend while others portions have a greater stiffness, for example, due to variation in a thickness of a wall of the passageway 208. This can be used to create chambers and/or other features of the passageway 208 to facilitate forming the aliquots.

In an example, the system includes a baseplate 215C. The baseplate 215C may be disposable or reusable. The baseplate 215 may provide rigidity to the system. The baseplate 215C may include support for the aliquot receptacles 111. The baseplate may be polymer, such as a polyurethane, polycarbonate, etc. The baseplate 215C may be metal, e.g., steel, aluminum, copper. The baseplate may include registration features to align with the layers 115A and 115B. For example, the baseplate 115C may include nobs and/or projections which fit into holes on the second layer 115B to facilitate alignment.

The baseplate 215C may be part of a mechanical device which includes the actuators 235, 237, and 239. The baseplate may be removable from the mechanical device, allowing loading of the layers 215B and 215A before placement in the mechanical device.

The input reservoir 202 may be formed by the first elastic layer 215A and second layer 215B. The input reservoir 202 receives the liquid to be aliquoted and/or components which will make that solution. In some examples, the liquid is mixed and/or homogenized while in the input reservoir 202. A gate and/or valve connecting the input reservoir 202 to the passageway 208 may be closed during mixing to allow greater pressures to be applied during mixing. The input reservoir 202 provides liquid to the passageway 208. The input reservoir 202 may be located off the first and second layers 115. The input reservoir 202 may provide liquid to the passageway 208 through a port and/or similar connection. In an example, the input reservoir 202 is a transfer tube. In another example, the transfer tube provides liquid to the input reservoir 202. The lid of the transfer tube may be pierced by two needles. The first needle is used to provide liquid to the transfer tube to solubilize and/or dilute any material in the transfer tube. The second needle receives the mixture and provides it to the passageway 208 and/or an input reservoir 202.

The output reservoir 203 may formed by the first elastic layer 215A and second layer 215B. The output reservoir 203 receives air and/or liquid from the distal end of the passageway 208. The output reservoir 203 contains the liquid used to clear the residual air from the passageway 208. Clearing residual air from the passageway allows each volume of the passageway 208 to contain the desired amount of liquid. This provides control to the volumes of the aliquots being formed and expressed into receptacles. It may be possible to minimize the size of the output reservoir or eliminate it entirely if no air is present in the passageways prior to filling with liquid.

The output reservoir 203 may be located off the layers 115. The output reservoir 203 may be connected to the passageway 208 by a valve, port, fitting, vent, and/or similar mechanism. Air and liquid expelled from the passageway 208 may be captured in the output reservoir 203. In an example, the liquid in the output reservoir 203 may be used retained as a control.

The passageway 208 connects the input reservoir 202 and the output reservoir 203. The liquid is flowed from the input reservoir 202 to the output reservoir 203. The first actuator 235 then closes portions of the passageway 208 forming volumes containing aliquots of liquid. The aliquots are isolated from each other. The aliquots are isolated from the input reservoir 202 and the output reservoir 203.

The first actuator 235 isolates volumes of the liquid filled passageway 208 from each other. Each isolated volume may be an aliquot. The first actuator 235 may isolate the volumes reversibly. The first actuator 235 may isolate the volumes irreversibly. For example, the first actuator 235 may mechanically press the elastic layer 115A against the second layer 115B then heat the area of the passageway 208 blocked to seal the passageway 208. In an example, the first actuator 235 actuates a mechanical latch that holds the passageway 208 closed, the latch may be molded into the baseplate 215C and/or the polymer films 215A, 215B. The latch may be provided as a disposable and/or reusable component which is pressed on top of the elastic layer 115A. The first actuator 235 may contain a heating element capable of welding the first 115A and second layers 115B, thereby sealing the liquid aliquots from one another. The heating element may be a resistive heater.

The second actuator 237 allows transfer of the liquid from the isolated volume to the receptacle. In an example, the second actuator 237 presses on the polymer film 215A above the isolated volume of liquid in the passageway 208. The liquid is forced out an opening in the passageway 208 and deposited in a receptacle. The opening may be a gate, valve, vent, etc. The receptacle 111 may be a well, a tube, a vial, and/or other container. The second actuator 237 may unblock the vent 205 and allow liquid to flow from the isolated volume. The liquid may flow under pressure and/or under recoil from the elastic first layer 215A.

The second actuator 237 may transfer all the liquid from the isolated volume. The second actuator 237 may transfer a portion of the volume. In an example, centrifuging is used to collect the aliquot in the bottom of the aliquot receptacle 111. For example, the device 300 may be subject a centrifuging to move the liquid to a desired location of the aliquot receptacle 111 for further testing.

The second actuator 237 may transfer an aliquot from each of the isolated volumes simultaneously. The aliquots may be transferred sequentially. The aliquots may be transferred into tubes, vials, wells, etc. All the aliquots may be transferred into tubes of a tube strip.

The third actuator 239 induces flow of the liquid from the input reservoir 202 into the passageway 208. In an example, the third actuator 239 presses on the elastic first layer over the input reservoir 202 to apply pressure to the liquid in the input reservoir 202 and induce flow. The third actuator 239 may operate a gate and/or valve between the input reservoir 202 and the passageway 208. The third actuator 239 may open the gate and/or valve to flow liquid from the input reservoir 202 to the passageway 208. The third actuator 239 may be a roller.

The system may further include a sensor. The sensor may detect the presence of liquid at a point in the system. The sensor may detect temperature. The sensor may be an electrical, optical, and/or other sensor. Information from the sensor maybe used to activate an actuator 235, 237, and/or 239. Multiple sensors may be used to facilitate automation of the aliquot forming process. The actuators and sensor may be operated by a controller which includes a processor and an associated memory containing instructions. Such components may facilitate automation of the associated activities.

The system 300 may further include a sealer. The sealer to seal connections between the isolated volumes and the receptacles 111 after the liquid has been transferred from the isolated volumes, wherein the sealer degrades nucleic acids to reduce contamination. The sealer may use mechanical pressure, blades, scissors, and/or similar mechanical components. The sealer may heat and melt/reflow a portion of thermoplastic, e.g., a thermoplastic layer 215. The sealer may apply chemicals, for example, oxidizers and/or a bleach, and/or radiation, for example, UV light.

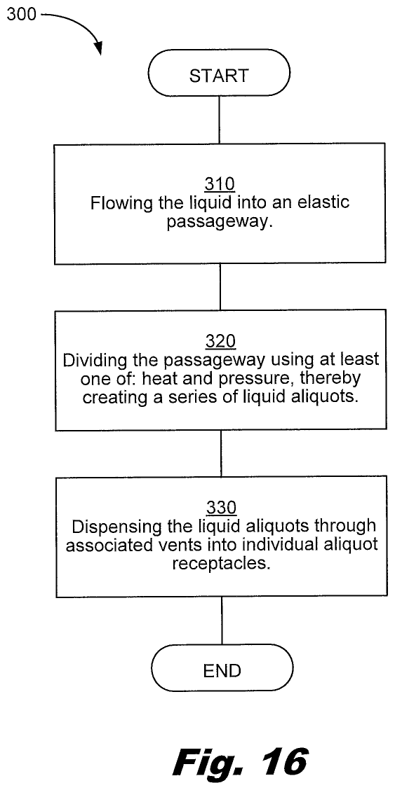

FIG. 16 shows a flowchart of a method 300 of separating a liquid containing nucleic material into aliquots, the method 300 including: flowing the liquid into an elastic passageway formed between two layers of material 310; dividing the passageway using at least one of: heat and pressure, thereby creating a series of liquid aliquots 320; and dispensing the liquid aliquots through associated vents into individual aliquot receptacles 330.

The method 300 of separating a liquid containing nucleic material into aliquots includes flowing the liquid into an elastic passageway 310. An elastic passageway has at least one wall composed of an elastic material. The elastic material may be an elastomer. The elastic material may be a polymer. The elastic material may be a composite. In an example, the elastic material has a recoverable elastic deformation of at least 50% (delta L/L).

The method 300 includes dividing the passageway using at least one of: heat and pressure, thereby creating a series of liquid aliquots 320. In an example, the passageway 8 is divided first by pressure and then sealed with heat, the heat melting a thermoplastic.

The method 300 includes dispensing the liquid aliquots through associated vents into individual aliquot receptacles 330. The vents may be opened to allow the aliquots to be dispensed. The vents may open when the pressure on the liquid aliquot increases. The layer above the liquid aliquot may be pressed and/or rolled to move the liquid of the aliquot. The individual receptacles may be wells of a well plate, PCR vials, and/or other receptacles to hold liquid.

The method 300 may further include: flowing the liquid into an input reservoir; mixing the liquid in the input reservoir; flowing the liquid into the passageway; and filling a portion of the passageway used to form the series of liquid aliquots with the liquid.

The method 300 may further include: piercing a transfer vessel; injecting the liquid into the transfer vessel; and transferring the liquid from the transfer vessel to the input reservoir.

The method 300 may further include: receiving a liquid aliquot through a vent into an aliquot receptacle; separating the aliquot receptacle and an attached portion of a distribution device from a different portion of the distribution device; and centrifuging the aliquot receptacle and the attached portion of the device to consolidate the aliquot of liquid.

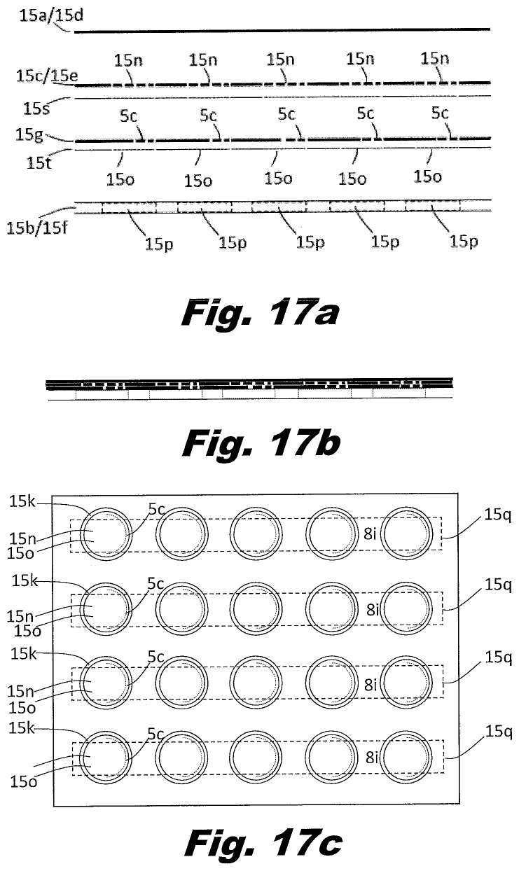

FIGS. 17 through 27 show an example of a system to interface with a well plate. In this example, the vent passageways 8 to individual wells are eliminated such that a single passageway 8 overlays a row or column of wells in the well plate. A rupturable membrane forms the portions of passageway that face the wells of the well plate. The vents are pre scored features in the rupturable membrane. For purposes of illustration, a 5×4 array is used, but the design is applicable to a variety of sizes and orientations of well plates including plates with a single row of wells, which are also known as tube strips.

FIGS. 17a through 17c shows the portion of the device that directly overlays the well plate. A lower, thin elastic layer 15c/15e is attached to a layer of adhesive 15s. A series of holes 15n matching the well spacing of the well plate 17 is formed through both layers. This elastic layer-adhesive assembly is thermally bonded to the upper elastic layer 15a/15d along the dashed lines 15q creating passageways 8i. The lower surface of layer 15c/15e is adhered to the upper surface of a rupturable membrane 15g using the layer of adhesive 15s. The holes 15n results in the upper portion of the rupturable membrane 15g being in contact with the lower portion of the upper elastic layer 15a/15d in the areas of the holes 15n. The adhesive does not extend into the areas of the holes 15n, such that fluid flow is not blocked by the adhesive. The rupturable membrane 15g is made from a thin and/or rigid material, which is more rigid than the elastic layer 15a/15d. A series of pre scored vents 5c are formed in the rupturable membrane 15g, to facilitate rupture at these locations. Rupture of the rupturable membrane 15g can be accomplished by pressing on the upper surface of elastic layer 15a/15d, which will stretch, enabling this force to be transferred to the pre-scored vents 5c, which will rupture. A second layer of adhesive 15t is positioned between the lower surface of the rupturable membrane 15g and a relatively thick layer of material 15b/15f. This layer of adhesive 15t also has holes 15o the same size and location relative to the well plate 17 as the holes 15n. FIG. 17b shows these layers laminated together via the adhesive layers and the thermal bonding process. FIG. 17c shows a top view of the layers laminated together as an assembly.

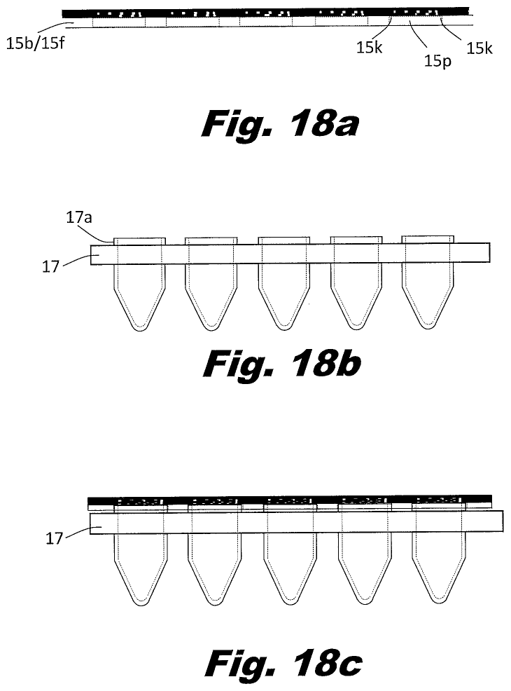

FIG. 18a-18c show an example of how the device can interface with a well plate utilizing the holes in the layer 15b/15f. In this example, layer 15b/15f is almost as thick as the height of the lips 17a of the well plate 17. For example, if the well plate 17 has a lip 17a that rises 0.020″ above the plate surface, layer 15f could be 0.018″ thick. Holes 15p are cut in layer 15b/15f to match the well pattern of the well plate 17 such that the holes 15p in layer 15b/15f will guide proper alignment of the device 15 to the well plate 17. The holes 15p are larger in diameter than the holes 15o in the lower adhesive layer 15t thereby exposing a donut-shaped ring of adhesive 15k on the underside of the rupturable membrane. In this manner, this portion of adhesive 15k contacts the top of the lip 17a thereby providing a seal of the device to the well plate.

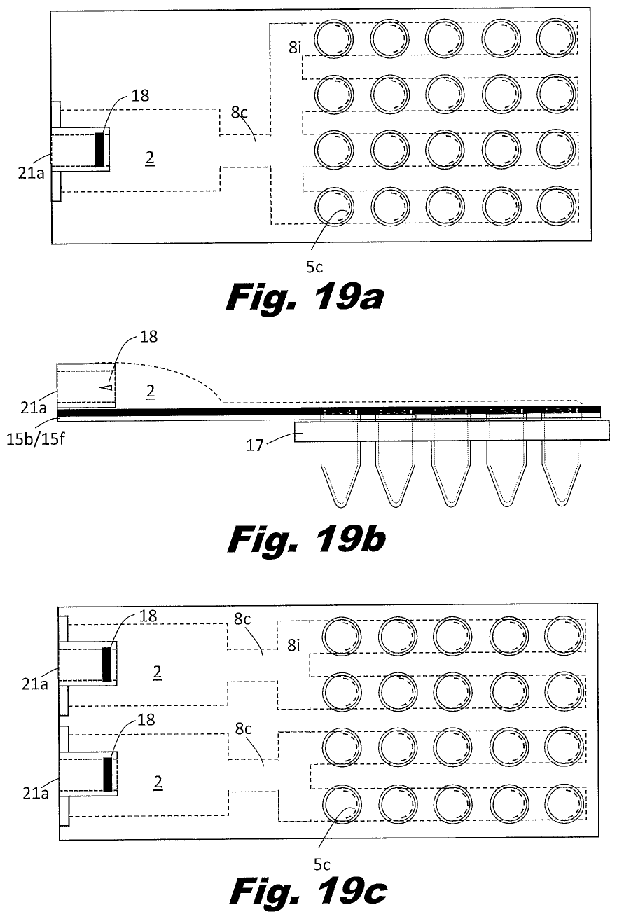

FIGS. 19a and 19b show an example of a complete device capable of interfacing with a well plate. In addition to the above described layers, this example contains a single input bladder 2 and no waste bladders. The input bladder 2 is created by welding the two elastic layers 15a/15d and 15c/15e and is contiguous with the passageways 8i. A passageway 8c connects the bladder 2 to the passageways 8i. The input bladder 2 contains a port 21a. The port 21a is sized to allow insertion of a sample-containing transfer vessel 12a. This transfer vessel 12a can be a standard PCR tube. Insertion of the sample-containing transfer vessel 12a plugs and seals the port 21a. The port 21a also contains a razor blade 18 or similar device to rupture the bottom of the tube 12a upon tube insertion, releasing the sample into the input bladder 2.

FIGS. 19c shows an example of a complete device with multiple ports 21a and multiple input bladders 2 and the associated channels 8c and 8i. This variation of the design allows the device to interface with multiple sample-containing transfer vessels 12a and transfer of the associated samples to different sets of wells in the same well plate.

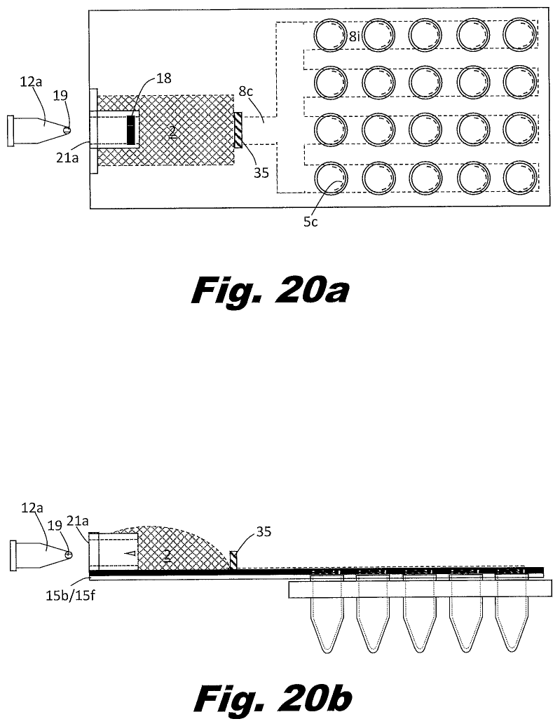

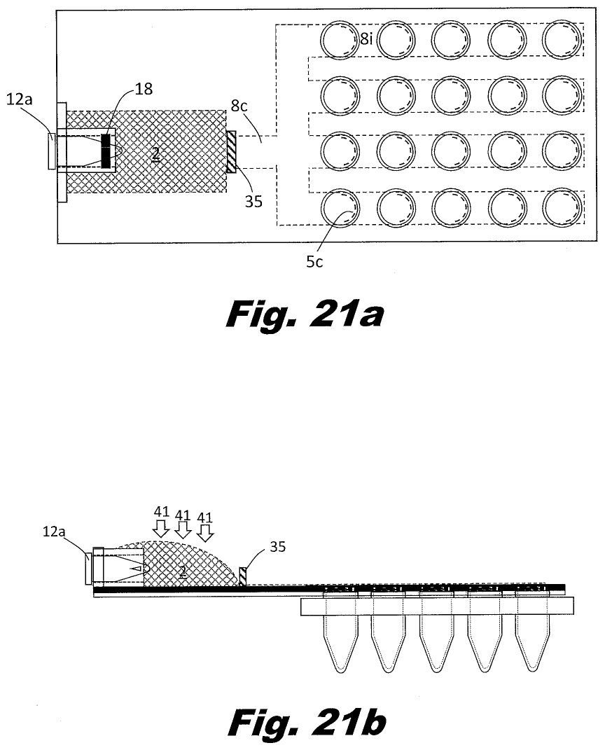

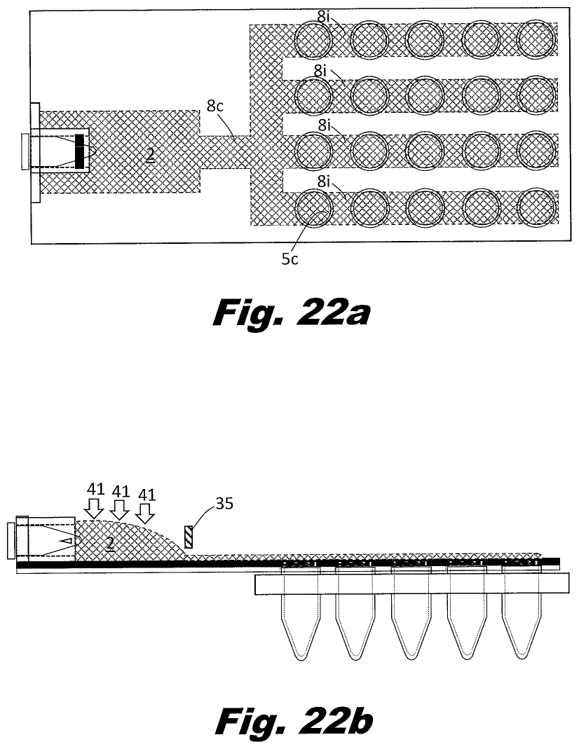

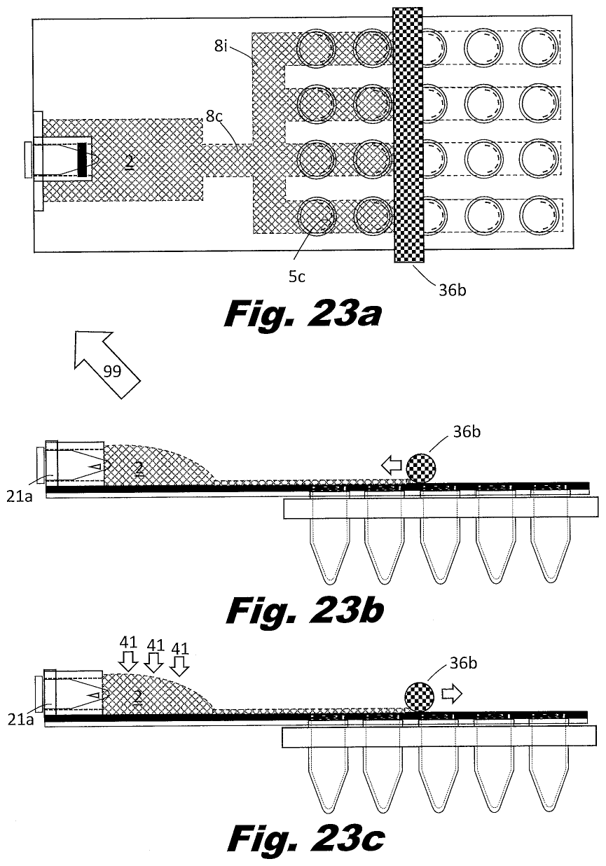

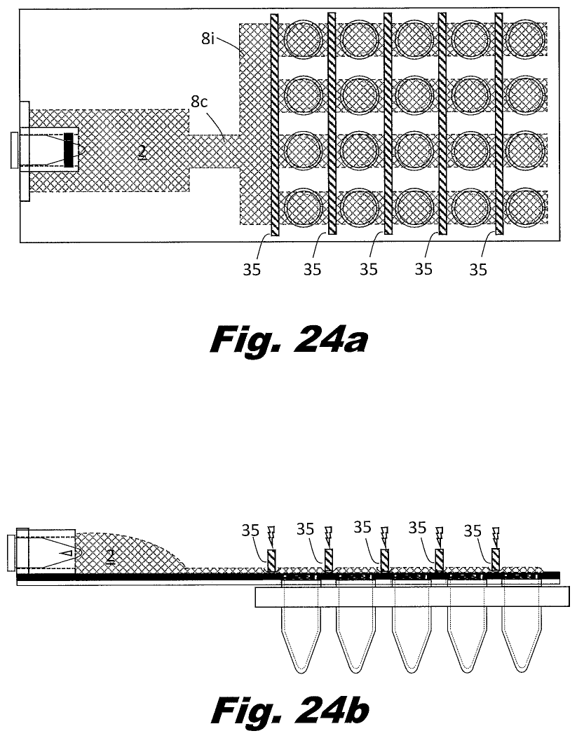

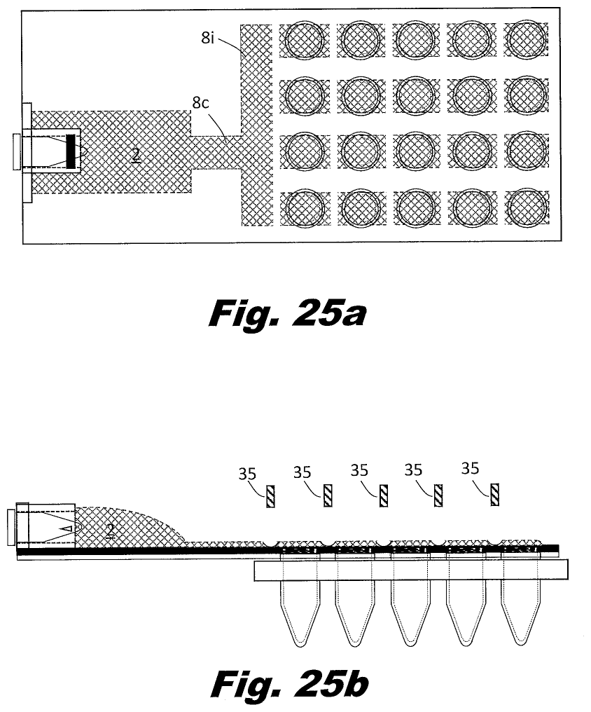

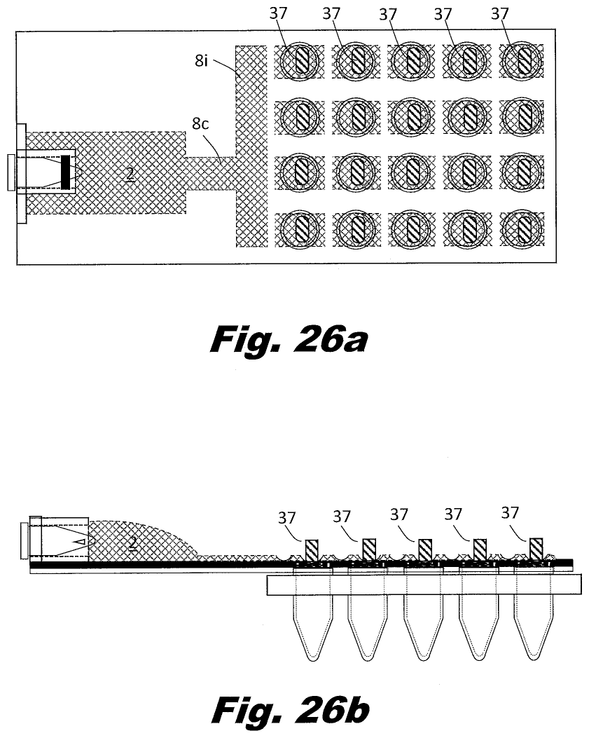

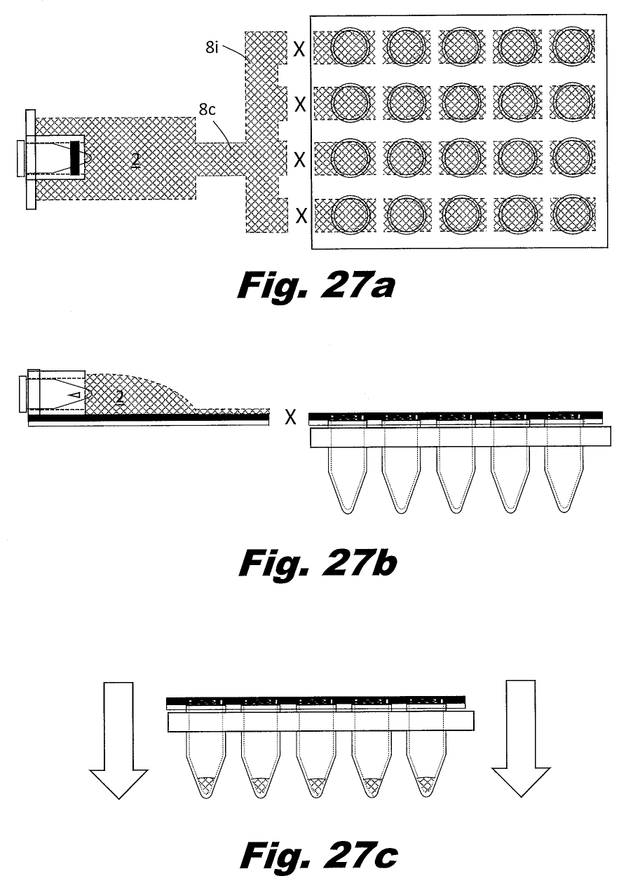

FIGS. 20a through 27c demonstrate use of the device shown in FIGS. 19a and 19b. Actuators 35 control fluid movement through the passageways 8 by pressing on the elastic upper surface 15a/15d of the passageways 8 reversibly sealing the passageway 8 against the lower surface 15c/15e of the passageway 8 in order to block flow. Also shown is a transfer vessel 12a containing a sample 19 that will be introduced into the device at the port 21a.

In FIGS. 20a and 20b, the passageway 8c is closed by the actuator 35. Then liquid, indicated with a cross hatch, is added directly to the input bladder 2 through the port 21a, using a device such as a pipette.

In FIGS. 21a and 21b, the transfer vessel 12a containing the sample 19 is pressed into the input port 21a. This action seals the port 21a and ruptures the bottom of the transfer vessel 12a on a razor blade 18 and/or other penetrator thereby releasing the sample 19 to be diluted into the liquid. Then downward force 41 can be applied and removed repeatedly from the elastic bladder 2 in order to mix the sample 19 with the liquid in the bladder 2.

In FIGS. 22a and 22b, downward pressure 41 is applied to the bladder 2 while the pressure on the actuator 35 is released. The downward pressure 41 then forces liquid into the passageways 8c and 8i.

In FIGS. 23a through 23c, a roller 36b pushes fluid back out of the passageways 8i back into the bladder 2. Any bubbles that were in the fluid are also moved into the bladder 2. The roller 36b utilizes a peristaltic effect by pressing on the elastic upper surface 15a/15d of the passageway 8i thereby sealing to the lower surface 15c/15e, then moving that seal along the passageways 8i to cause movement of the fluid within the passageways 8i. In normal use, the device in FIG. 23b is held in a partially vertical (i.e., non-horizontal) orientation (e.g., where vertical is indicated by arrow 99) such that bubbles that enter the bladder 2 rise to the port 21a side of the bladder 2. In other examples, the bladder 2 is configured to have an upper surface which slopes away from the entrance to the passageway 8i. The bladder 2 having a sloped upper surface helps trap gas bubbles away from an entrance to the passageway 8. In an example, the sloped surface is formed by rotating the assembly as shown by the arrow 99 in FIG. 23b. In FIG. 23c, the roller 36b then reverses direction and pressure 41 is reapplied to the bladder 2, which forces the liquid back into the passageways 8i while leaving/trapping the bubbles in the bladder. Repeated cycles of the actions shown in FIGS. 23b and 23c remove bubbles from the passageways 8c and 8i. These actions may also mix the fluid in the passageways 8c and 8i, which can potentially avoid differences in liquid composition that may exist when the fluid first enters the passageways 8c and 8i.

In FIGS. 24a and 24b, a series of parallel actuators 35 press on the passageways 8i. Because the passageways 8i contain fluid, the actuators 35 divide the passageways 8i into aliquots of fluid. Heat is then applied to the actuators 35, for example, as an electrical current, in order to thermally bond layer 15a/15d to layer 15c/15e and lock the fluid in the aliquots formed from the passageways 8i. The fluid volume of the aliquots is controlled by a number of factors, including: 1) the amount of pressure 41 placed on the bladder 2, which directly effects the amount of swelling of the passageways 8i formed by the elastic layers 15a/15d to 15c/15e and 2) the dimensions of the passageways 8i created by thermally bonding 15q layers 15a/d and 15c/e.

In FIGS. 25a and 25b, the actuators 35 are released showing the aliquots of fluid formed by the thermal bonding process described above.

In FIGS. 26a and 26b, a series of actuators 37 press downward on layer 15a/15d directly above the aliquots. Elastic layer 15a/15d stretches but the rupturable membrane 15g below it is more rigid and therefore ruptures at the vents 5c.

In FIGS. 27a and 27b, the portion of the device 15 containing the bladder 2 is cleaved (indicated with the letters “X”) along one of the thermal bonded lines created in FIGS. 24a and 24b. Cleavage X is performed using a device such as blade or laser. Residual sample, which can be a PCR product, that could be exposed at the cleavage site can be avoided if the thermal bonding process is performed for a sufficient time to chemically degrade the sample in the area of passageway 8i indicated by X, and/or if the post cleavage interface indicated by X is chemically treated.

In FIG. 27c, the well plate portion of the device along with the associated well plate, is subject to centrifugal force in order to move the liquid from the aliquots described above into the bottom of the wells.

Elements from the various examples may be mixed and matched to obtain a desired system. For example, the secondary port 31 for the dilutant from FIG. 5 may be readily integrated into FIG. 1. The number of reservoirs 1, 2, 3 and input ports 21 may be modified to reflect different sample preparation needs. It will be appreciated that, within the principles described by this specification, a vast number of variations exist. It should also be appreciated that the examples described are only examples, and are not intended to limit the scope, applicability, or construction of the claims in any way.

What is claimed is:

1. A device for forming a liquid aliquot, the device comprising:
a first layer;
an elastic second layer overlapping the first layer;
a passageway to receive and hold a volume of liquid, the passageway formed from the first and second layers;
a first actuator to press on the elastic layer thereby dividing the liquid filled passageway into a series of liquid aliquots;
a series of vents associated with the series of aliquots; and
a second actuator to control flow of liquid aliquots through the associated vents.

2. The device of claim 1, wherein the first actuator contains a heating element capable of welding the first and second layers, thereby sealing the liquid aliquots from one another.

3. The device of claim 1, further comprising a rupturable membrane, wherein rupturing of the membrane controls flow of a liquid aliquot through the associated vent.

4. The device of claim 3, wherein the rupturable membrane comprises scored locations to rupture when pressure is applied to an associated aliquot.

5. The device of claim 1, wherein the second actuator is a presser, wherein pressing the second elastic layer toward the rupturable membrane ruptures the rupturable membrane thereby controlling the flow of a liquid aliquot through the associated vent.

6. The device of claim 1, further comprising an input reservoir to provide liquid to the passageway.

7. The device of claim 6, further comprising a port associated with the input reservoir, the port capable of interfacing with a transfer vessel.

8. The device of claim 6, wherein the device comprises a plurality of input reservoirs, each containing a port capable of interfacing with a transfer vessel.

9. A method of separating a liquid containing nucleic material into aliquots, the method comprising:
flowing the liquid into an elastic passageway;
dividing the passageway using pressure, thereby creating a series of liquid aliquots; and
dispensing the liquid aliquots through associated vents into individual aliquot receptacles.

10. The method of claim 9, further comprising:
applying pressure to the series of liquid aliquots in order to rupture rupturable membranes covering the vents.

11. The method of claim 9, further comprising:
flowing the liquid into an input reservoir;
mixing the liquid in the input reservoir;
flowing the liquid into the passageway;
and filling a portion of the passageway used to form the series of liquid aliquots with the liquid.

12. The method of claim 11, wherein flowing the liquid into the passageway comprises compressing the input reservoir.

13. The method of claim 11, further comprising pushing liquid from the passageway back into the input reservoir from the passageway in order to remove bubbles from the passageway, and compressing the input reservoir to refill the passageway with the liquid.

14. The method of claim 13, wherein an upper surface of the input reservoir is sloped relative to horizontal so as to displace bubbles from proximate to the entrance to the passageway.

15. A system for preparing a liquid aliquot, the system comprising:
an elastic passageway, the passageway connected at one end to an input reservoir;
a third actuator to control the flow of a liquid from the input reservoir into the passageway;
a first actuator to divide the passageway into a plurality of liquid aliquots; and
a second actuator to control the flow of a liquid aliquot from an isolated portion of the passageway through a vent.

16. The system of claim 15, further comprising an input port that fluidically connects a transfer vessel to the input reservoir.

17. The system of claim 17 further comprising a piercing device associated with the input port capable of piercing a transfer vessel thereby allowing transfer of the liquid from the transfer vessel into to the input reservoir.

18. The system of claim 15, further comprising:
a sealer to irreversibly seal a portion of the passageway; and
a cutter to cleave the sealed portion of the passageway and thereby separate the aliquot receptacle and an associated containment portion of the device from the remainder of the device,
wherein nucleic acids present at cleaved surfaces of the sealed portion are degraded using at least one of: heat, chemical treatment, ionizing radiation, and non-ionizing radiation.

19. The system of claim 18, where the nucleic acids present at the cleaved surfaces of the sealed portions are degraded using heat, wherein the heat is applied for longer than is required to seal and cut the portion of the passageway.

20. The system of claim 15, wherein the vent comprises a scored layer of material, the scored layer of material having a greater rigidity than the elastic passageway such that compressing the aliquot causes the scored layer of material to rupture.
